(19) United States
(12) Patent Application Publication
(10) Pub. No.: US 2023/0395187 A1
(43) Pub. Date: Dec. 7, 2023

(54) SYSTEMS AND METHODS FOR DE NOVO DESIGN OF PROTEIN INTERACTIONS WITH LEARNED SURFACE FINGERPRINTS

(71) Applicants: ÉCOLE POLYTECHNIQUE FÉDÉRALE DE LAUSANNE (EPFL), Lausanne (CH); IMPERIAL COLLEGE INNOVATION LIMITED, London (GB)

(72) Inventors: Pablo Gainza Cirauqui, Lausanne (CH); Sarah Wehrle, Lausanne (CH); Alexandra Van Hall-Beauvais, Lausanne (CH); Anthony Marchand, Lausanne (CH); Andreas Scheck, Lausanne (CH); Zander Harteveld, Lausanne (CH); Freyr Sverrisson, St. Sulpice (CH); Michael Bronstein, London (GB); Bruno Emanuel Ferreira De Sousa Correia, Saint-Saphorin-Sur-Morges (CH)

(21) Appl. No.: 18/206,873

(22) Filed: Jun. 7, 2023

(30) Foreign Application Priority Data
Jun. 7, 2022 (EP) 22177692.5

(51) Int. Cl. G16B 15/30; G16B 20/30; G16B 40/20

(52) U.S. Cl. CPC G16B 15/30; G16B 20/30; G16B 40/20

(57) ABSTRACT

The present application relates to a computer-implemented systems and methods for protein interaction design using surface fingerprints. The method comprises predicting at least one target interface site with high binding propensity, wherein, optionally, the step of predicting at least one target buried interface site comprises generating at least one surface fingerprint associated with a protein interaction based on at least one protein interface, wherein the at least one surface fingerprint preferably embeds geometric and/or chemical features of molecular surfaces, identifying at least one binding seed that displays required features to engage the target site, and performing a binding seed transplantation to protein scaffolds to confer stability and additional contacts on the designed interface.

### CROSS REFERENCE TO RELATED APPLICATION(S) AND PUBLICATION(S)

This application claims the benefit of European Application No. EP22177692 (filed on Jun. 7, 2022). The entirety of the foregoing application is incorporated by reference herein.

In addition, this application shares at least one common inventors with those individuals named as authors of the following publication: Gainza et al., “DE NOVO DESIGN OF PROTEIN INTERACTIONS WITH LEARNED SURFACE FINGERPRINTS,” Nature vol. 617 (May 4, 2023). The entirety of the foregoing publication is incorporated by reference herein.

### BACKGROUND

Designing novel protein-protein interactions (PPIs) remains a fundamental challenge in computational protein design, with broad basic and translational applications in biology. The challenge consists of generating amino acid sequences that engage a target site and form a quaternary complex with a given protein, a stringent test of our understanding of the determinants that drive biomolecular interactions. Robust computational methods to design de novo PP Is could be used to rapidly engineer protein-based therapeutics, as antibodies and inhibitors, vaccine design and others, and therefore are of major interest for biomedical and translational applications.

Despite recent advances in rational PPI design and prediction, designing novel protein binders against specific targets remains a challenge, particularly when no structural elements from preexisting binders are known. Current state-of-the-art methods for de novo PPI design, such as hotspot-centric approaches and rotamer information fields, rely on placing disembodied residues on the target interface and then optimizing their presentation on a protein scaffold. Intrinsic limitations of these approaches relate to the very weak energetic signatures provided by scoring functions to single-side chain placements, which is compounded in flat interfaces that lack deep pockets. These methods also face the challenge of finding compatible protein scaffolds to precisely display the generated constellations of residues. To circumvent these limitations, new approaches are needed to design de novo binders to various surface types and protein sites.

A long-standing model of molecular recognition postulates that PPIs form between protein surfaces with chemical and geometric complementarity. The complementarity features arise as a consequence of the energetic contributions that are critical to stabilize PPIs, including van der Waals interactions (geometric complementarity), hydrophobic effect, and electrostatics interactions (chemical complementarity). At the structural level, most protein interfaces contain surface regions that become inaccessible to solvent upon complex formation, which we refer to as buried or core interface, as well as patches that are involved in the interface but also exposed to solvent, which we refer as the interface rim. Residues within the buried areas tend to be much less tolerant of mutations and give larger contributions to the affinity of PP Is. Rim regions are often more polar and tolerant to mutations, giving also important contributions to affinity and more notably specificity. Based on these general principles of molecular recognition, we introduce a novel protein design approach based on the critical importance of the fully buried patches of the interface to drive protein interactions. We implemented these design principles by leveraging surface fingerprints learned from interacting protein surfaces which capture features that are determinant for molecular recognition.

### SUMMARY

Physical interactions between proteins are essential for most biological processes governing life. However, the determinants of such interactions have been challenging to understand, even as genomic, proteomic, and structural data grows. This knowledge gap has been a major obstacle for the complete understanding of cellular protein-protein interaction (PPI) networks and for the de novo design of protein binders that are crucial for synthetic biology and translational applications. We exploit a geometric deep learning framework to generate surface fingerprints from protein structures, which are learned from protein interfaces to describe geometric and chemical features critical to drive PPIs. We hypothesized these fingerprints efficiently capture key aspects of molecular recognition, and used them as the foundation of a new PPI design method. As a proof-of-principle, we computationally designed four de novo protein binders to engage three protein targets: SARS-CoV-2 spike, PD-1, and PD-L1. The designs bound the target sites with high affinity upon experimental optimization, structural and mutational characterization showed highly accurate predictions. Overall, we present a surface-centric approach to describe structure and capture molecular recognition determinants enabling a novel approach for the de novo design of protein interactions and, more broadly, of artificial proteins with function.

### BRIEF DESCRIPTION OF THE DRAWINGS

Pursuant to 37 C.F.R. § 1.84(a)(2), the patent or application file contains at least one drawing executed in color. Copies of this patent or patent application publication with color drawing(s) will be provided by the Office upon request and payment of the necessary fee.

The Figures described below depict various aspects of the system and methods disclosed therein. It should be understood that each Figure depicts an embodiment of a particular aspect of the disclosed system and methods, and that each of the Figures is intended to accord with a possible embodiment thereof. Further, wherever possible, the following description refers to the reference numerals included in the following Figures, in which features depicted in multiple Figures are designated with consistent reference numerals.

There are shown in the drawings arrangements which are presently discussed, it being understood, however, that the present embodiments are not limited to the precise arrangements and instrumentalities shown, wherein:

FIG. 1 illustrates an example surface-centric design of protein interactions and related systems and methods in accordance with various embodiments disclosed herein.

FIG. 2 illustrates an example design and optimization of a SARS-CoV-2 RBD binder and related systems and methods in accordance with various embodiments disclosed herein.

FIG. 3 illustrates an example design and optimization of PD-L1 binder designs and related systems and methods in accordance with various embodiments disclosed herein.

FIG. 4 illustrates an example optimized workflow and de novo binders for PD-1 and related systems and methods in accordance with various embodiments disclosed herein.

FIG. 5 illustrates an example overview of the neural network architectures used in the MaSIF protocols and related systems and methods in accordance with various embodiments disclosed herein.

FIG. 6 illustrates an example modeling buried surfaces as radial patches and related systems and methods in accordance with various embodiments disclosed herein.

FIG. 7 illustrates an example benchmarking the identification of helices based on patches and related systems and methods in accordance with various embodiments disclosed herein.

FIG. 8 illustrates an example MaSIF-site target site prediction on SARS-CoV-2 RBD, PD-L1, and PD-1 and related systems and methods in accordance with various embodiments disclosed herein.

FIG. 9 illustrates an example RBD-binder metrics for up- and down-orientation and related systems and methods in accordance with various embodiments disclosed herein.

FIG. 10 illustrates an example original RBD-binder designs displayed on yeast and related systems and methods in accordance with various embodiments disclosed herein.

FIG. 11 illustrates an example directed library for DBR3_01 and related systems and methods in accordance with various embodiments disclosed herein.

FIG. 12 illustrates an example SPR curves for DBR3 designs and related systems and methods in accordance with various embodiments disclosed herein.

FIG. 13 illustrates an example SSM of DBR3_02 and related systems and methods in accordance with various embodiments disclosed herein.

FIG. 14 illustrates an example biophysical characterization of the designed binders and related systems and methods in accordance with various embodiments disclosed herein.

FIG. 15 illustrates an example cryo-EM structure of DBR3_03 and related systems and methods in accordance with various embodiments disclosed herein.

FIG. 16 illustrates an example GSFSC resolution mapping of RBD-binders and NTD-binders and related systems and methods in accordance with various embodiments disclosed herein.

FIG. 17 illustrates an example spike-D614G and helix binders and related systems and methods in accordance with various embodiments disclosed herein.

FIG. 18 illustrates an example DBR3_03 binds to several variants and related systems and methods in accordance with various embodiments disclosed herein.

FIG. 19 illustrates an example cryo-EM structure of DBR3_03 and related systems and methods in accordance with various embodiments disclosed herein.

FIG. 20 illustrates an example GSFSC resolution mapping of RBD-binders and NTD-binders and related systems and methods in accordance with various embodiments disclosed herein.

FIG. 21 illustrates an example spike-omicron and binder/receptor-binding motif (RBM) interface and related systems and methods in accordance with various embodiments disclosed herein.

FIG. 22 illustrates an example planarity of the predicted targeted interfaces and related systems and methods in accordance with various embodiments disclosed herein.

FIG. 23 illustrates an example clusters of binding seeds docked on the PD-L1 surface and related systems and methods in accordance with various embodiments disclosed herein.

FIG. 24 illustrates an example binding signals of initial PD-L1 binder designs and related systems and methods in accordance with various embodiments disclosed herein.

FIG. 25 illustrates an example composition and outcome of yeast display libraries and related systems and methods in accordance with various embodiments disclosed herein.

FIG. 26 illustrates an example complete SSM library of DBL1_03 and related systems and methods in accordance with various embodiments disclosed herein.

FIG. 27 illustrates an example overview of DBL2_03 SSM library and related systems and methods in accordance with various embodiments disclosed herein.

FIG. 28 illustrates an example overview and comparison between PD-1 binders and related systems and methods in accordance with various embodiments disclosed herein.

FIG. 29 illustrates an example competition and specificity binding assay of DBP13_01 and DBP40_01 on the surface of yeast and related systems and methods in accordance with various embodiments disclosed herein.

FIG. 30 illustrates an example SSM of DBP13_01 and related systems and methods in accordance with various embodiments disclosed herein.

FIG. 31 illustrates an example SSM of DBP13_01 and related systems and methods in accordance with various embodiments disclosed herein.

FIG. 32 illustrates an example surface comparison between seeds, designs and final/predicted structures and related systems and methods in accordance with various embodiments disclosed herein.

FIG. 33 illustrates an example surface similarity between the residues corresponding to the initial design and the final binding seed and related systems and methods in accordance with various embodiments disclosed herein.

The Figures depict preferred embodiments for purposes of illustration only. Alternative embodiments of the systems and methods illustrated herein may be employed without departing from the principles of the invention described herein.

### DETAILED DESCRIPTION

Design of De Novo PPIs Using Learned Surface Fingerprints.

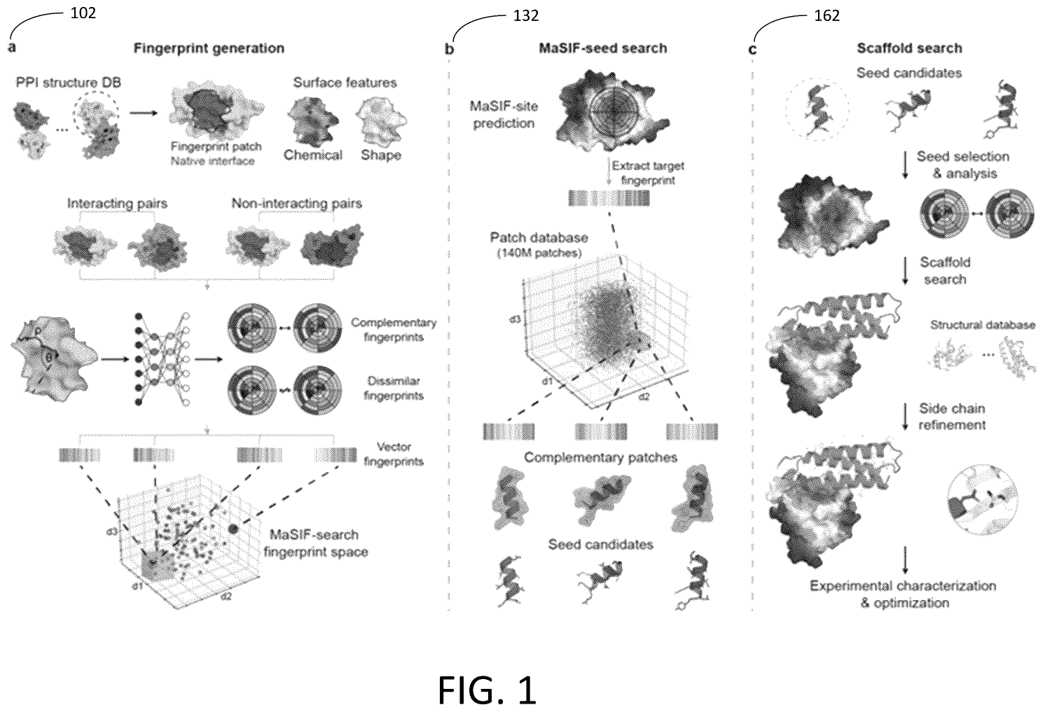

We have introduced a geometric deep learning framework, MaSIF (Molecular Surface Interaction Fingerprinting), to generate surface fingerprints that embed the geometric and chemical features of molecular surfaces, and learn patterns that determine the propensity of protein interactions. Since MaSIF has robust performance in PPI-related prediction tasks, we hypothesized that we could leverage it to design novel PPIs by targeting sites only using structural information from the target. To address the de novo PPI design problem we devised a three-stage computational approach (FIG. 1): I) prediction of target buried interface sites with high binding propensity using MaSIF-site (FIG. 1a); II) surface fingerprint-based search for short structural motifs (binding seeds) that display the required features to engage the target site, a protocol we refer to as MaSIF-seed (FIG. 1a,b); Ill) binding seed transplantation to protein scaffolds to confer stability and additional contacts on the designed interface (FIG. 1c) using established motif transplantation techniques.

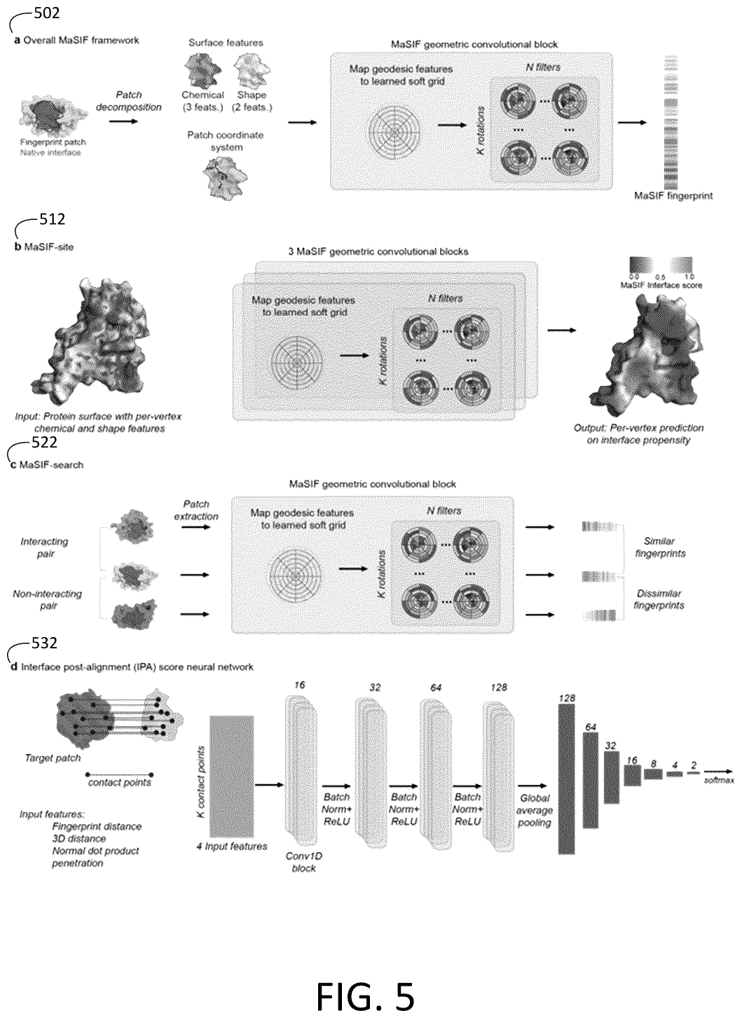

The new MaSIF-seed protocol tackles the problem of identifying binding seeds that can mediate productive binding interactions (see FIG. 1, FIG. 5). This task stands as a remarkable challenge in protein design due to the extensive space of structural possibilities to explore, as well as the required precision given that subtle atomic-level changes, such as misplaced methyl groups, uncoordinated water molecules in the interface, or incompatible charges, are sufficient to disrupt PPIs.

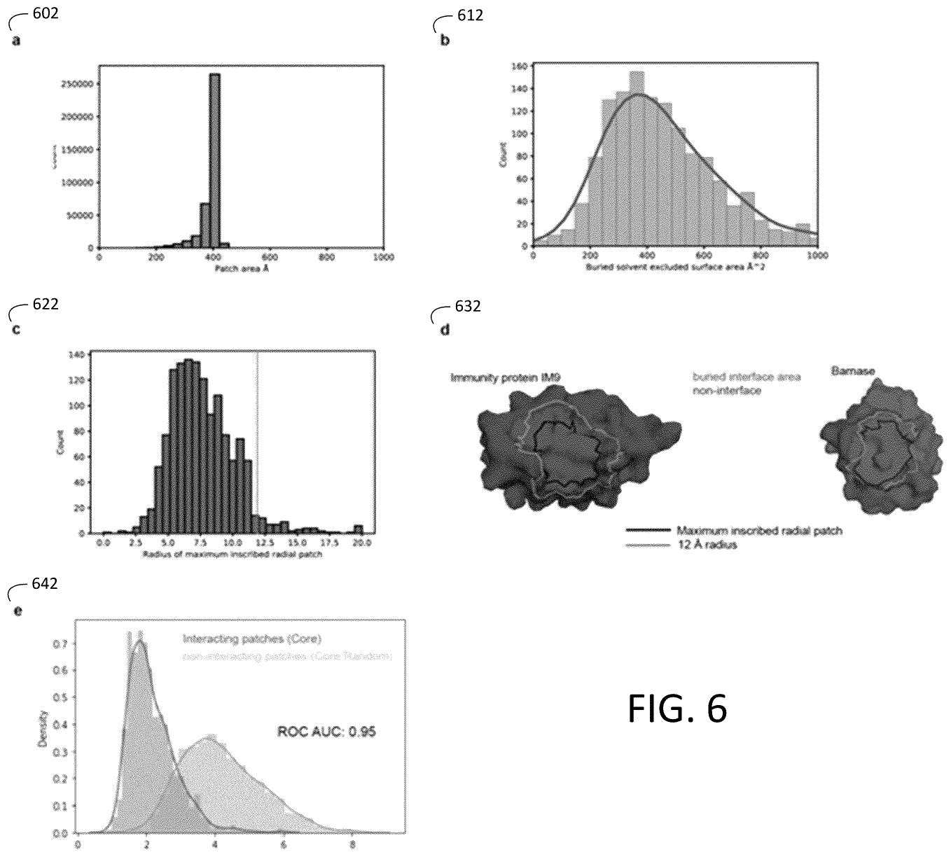

In MaSIF-seed, protein molecular surfaces are decomposed into overlapping radial patches with a 12 Å radius, capturing on average nearly 400 Å² of surface area, consistent with the buried surface areas observed in native interfaces (FIG. 6). For each point within the patch we compute chemical and geometric features, as well as a coordinate system in geodesic space to locate points within the patch relative to each other. A neural network is trained to output vector fingerprint descriptors that are complementary between interacting protein pairs and dissimilar between non-interacting pairs (FIG. 1a, FIG. 5). Matched surface patches are aligned to the target site and scored with a second neural network, outputting an interface post-alignment (IPA) score to further improve the discrimination performance of the surface descriptors (see Methods).

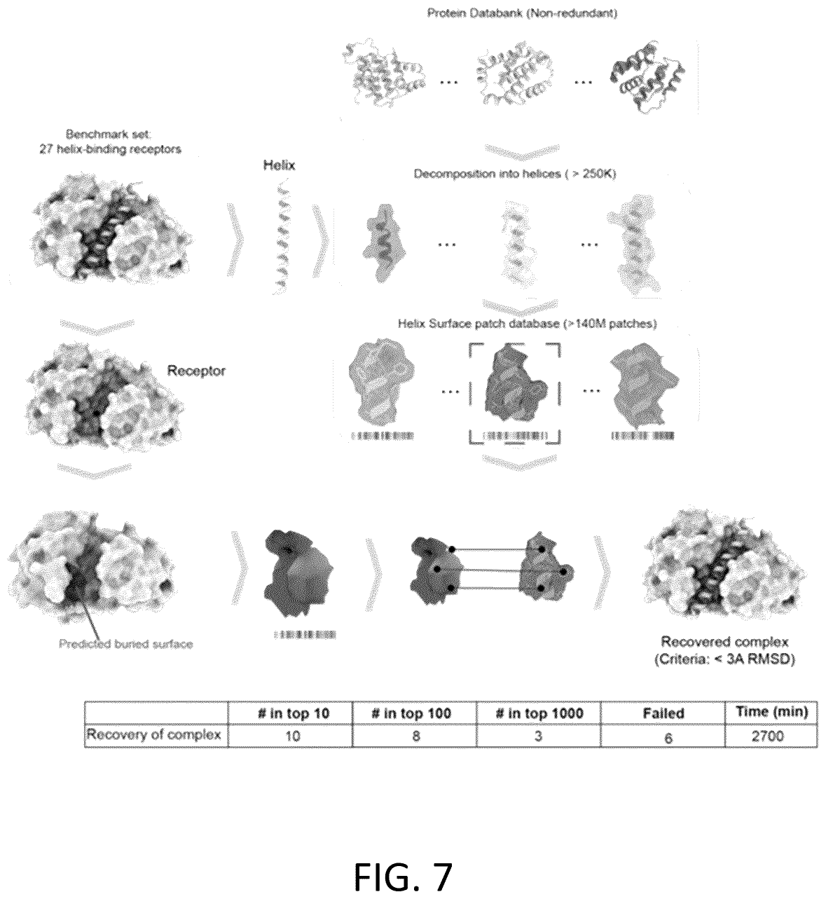

To benchmark our method, we built a database of 31 dimeric PPIs from the PDBBind database, where, in each pair, the interface of one of the proteins consists of a single α-helical domain. We then assembled a database of 1000 helical fragments (decomposed into 600K patches for MaSIF-seed) extracted randomly from the PDB, and benchmarked the capacity of MaSIF-seed to identify the true binder from the co-crystal structure in the correct orientation (<3 Å iRMSD) among 1000 decoys (FIG. 7). In 22 of the 31 cases, MaSIF-seed identified the native helix in the correct orientation as the top result, and in 24 out of 31 in the top 10, with an average time of 15 minutes per receptor. We benchmarked MasSIF-seed to a set of fast, well-established docking methods (Table 1). By comparison, the best performing method, ZDock+ZRank2 identified only 19 in the top 100 and 5 as top results, with an average running time of 2946 minutes, approximately 200 times slower than MaS IF-seed.

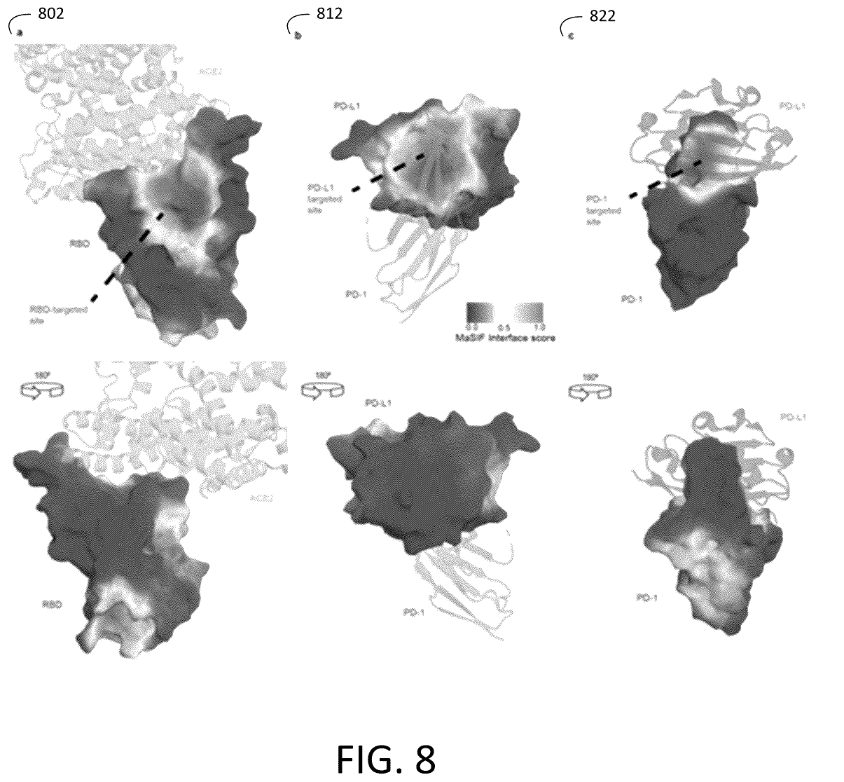

Encouraged by MaSIF-seed's speed and accuracy in discriminating the true binders from decoys based on rich surface features, we sought to design de novo protein binders to engage challenging protein targets in disease-relevant sites. We thus assembled a database of 250,000 structural fragments (140 million surface fingerprints) with helical secondary structure, extracted from the PDB. We computationally designed and experimentally validated binders against three structurally diverse targets: the receptor binding domain (RBD) of the SARS-CoV-2 spike protein where we identified a neutralization-sensitive site, and the two partners of the PD-1/PD-L1 complex, an important protein interaction in immuno-oncology that displays a flat interface and is considered a “hard-to-drug” target using small-molecules (FIG. 8).

Table 1 is shown below, which shows an example benchmark of MaSIF-seed against traditional docking methods in recovering the native binder in the correct conformation from co-crystal structures for 31 helix-receptor complexes, discriminating between 1000 decoys.

TABLE 1

| | # in | # in | # in | | Avg time |
|---|---|---|---|---|---|
| Method | top 1 | top 10 | top 100 | >100 | (m) |
| MaSIF-seed | 18 | 18 | 20 | 11 | 15 |
| PatchDock + MaSIF-site | 3 | 5 | 11 | 20 | 86 |
| ZDOCK | 3 | 4 | 8 | 23 | 2715 |
| ZDOCK + MaSIF-site | 1 | 6 | 10 | 21 | 2485 |
| ZDOCK + ZRank2 | 6 | 12 | 21 | 9 | 2946 |
| ZDOCK + Zrank2 + | 5 | 11 | 19 | 12 | 2710 |
| MaSIF-site | | | | | |

With reference to Table 1, the first column identifies the benchmarked method. The second through fourth columns identify the number of receptors for which the given benchmarked method recovered the native binding helix (<3 Å iRMSD) within the top 1 (second column), top 10 (third column), and top 100 (fourth column) results. Finally, the fifth columns shows the average running time of the given benchmarked method in minutes, excluding precomputation time.

FIG. 1 illustrates example surface-centric design of protein interactions and related systems and methods in accordance with various embodiments disclosed herein. As shown for FIG. 1, a fingerprint generation procedure or step 102 (section a) is executed or otherwise implemented (e.g., by one or more processors), where as shown for FIG. 1, protein binding sites are spatially embedded as vector fingerprints. At step 102 protein surfaces are decomposed into overlapping radial patches and a neural network trained on native interacting protein pairs to learn to embed the fingerprints such that complementary fingerprints are placed in a similar region of space, represented here using PCA to visualize a subsample of the fingerprint space. A green box shows the location in space of complementary fingerprints.

Further, as shown for FIG. 1, a fingerprint generation procedure or step 132 (section b) is executed or otherwise implemented (e.g., by one or more processors), where as shown for FIG. 1, new binding seeds are identified. At step 132, a target patch is automatically identified by MaSIF-site based on the propensity to form buried interfaces. Using MaSIF-seed search, a fingerprint is then computed on this patch and all complementary fingerprints in a large database (˜140M patches) are compared. A short list of complementary patches is selected and the fingerprints are used to align and rescore patches resulting in a set of seed candidates.

Further, as shown for FIG. 1, a fingerprint generation procedure or step 162 (section c) is executed or otherwise implemented (e.g., by one or more processors), where as shown for FIG. 1, seed candidates are transferred and/or designs are generated, developed, and/or otherwise identified. At stop 162, the selected seed is transferred to a protein scaffold and the rest of the interface is redesigned. The top designs are selected and tested experimentally using yeast display.

Targeting a Predicted Neutralization-Sensitive Site on SARS-CoV-2 Spike Protein.

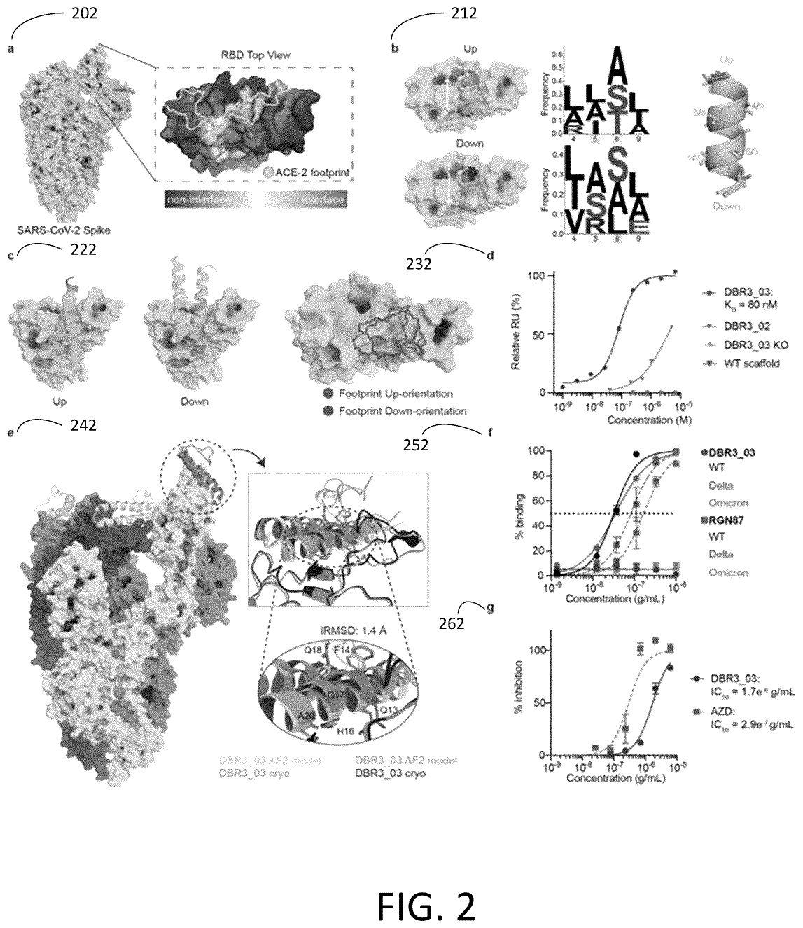

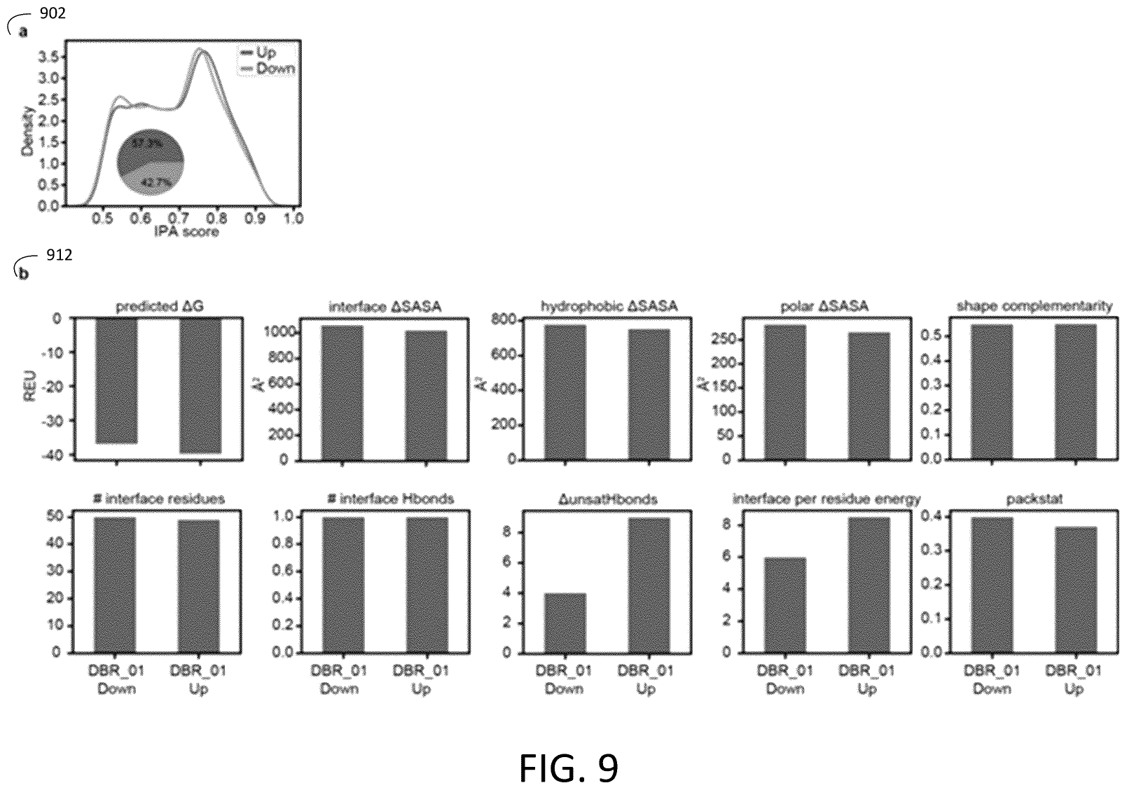

We applied our surface-centric approach to design de novo binders to target the SARS-CoV-2 RBD. First, we used MaSIF-site to predict surface sites on the RBD with high propensity to be engaged by protein binders. We selected a site distinct from the ACE2 binding region, but sufficiently proximal that a putative binder could inhibit the ACE2-RBD interaction (FIG. 2a). At the time, there were no known binders to this site. We searched our database of 140 million surface fingerprints derived from helical fragments to find binding seeds that could target the selected site. The 7713 binding seeds MaSIF-seed provided showed two prominent features: I) a contact surface absent of residues with strong binding hotspot features (e.g. large hydrophobic residues); II) an equivalent distribution of binding seeds in two distinct orientations of the helical fragment, with the seeds binding at 180° from each other (FIG. 2b), hinting that both binding modes are plausible. Remarkably, both orientations of the binding seeds present very similar signatures at the surface fingerprint level (FIG. 9a) and at the sequence level (FIG. 2b).

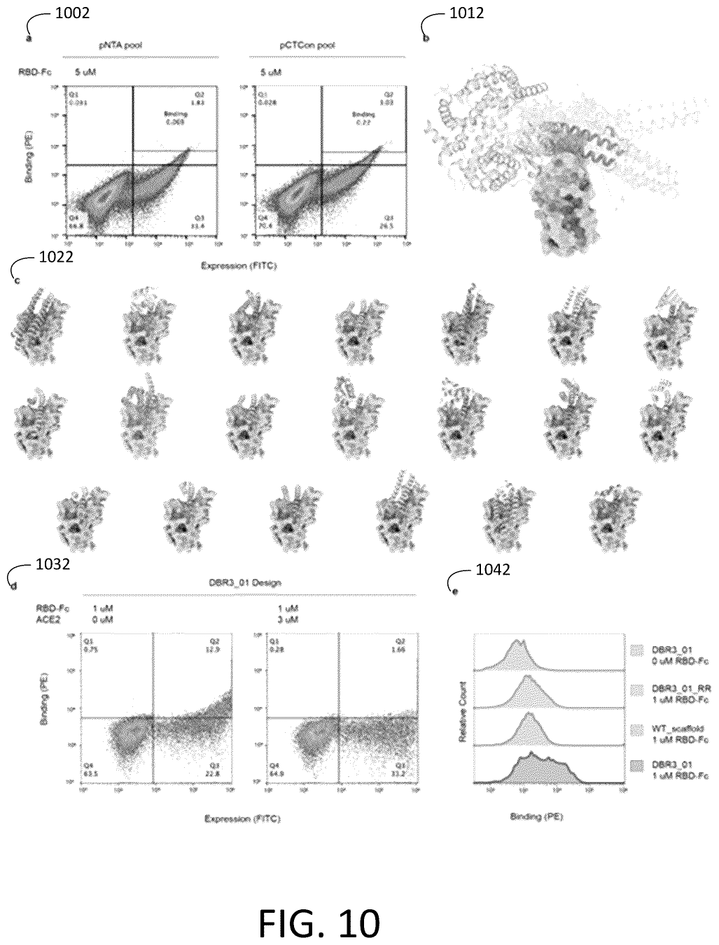

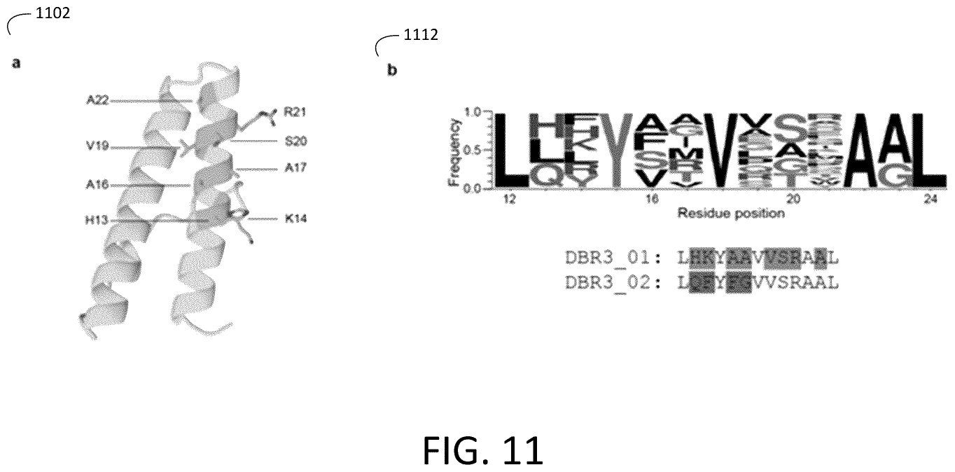

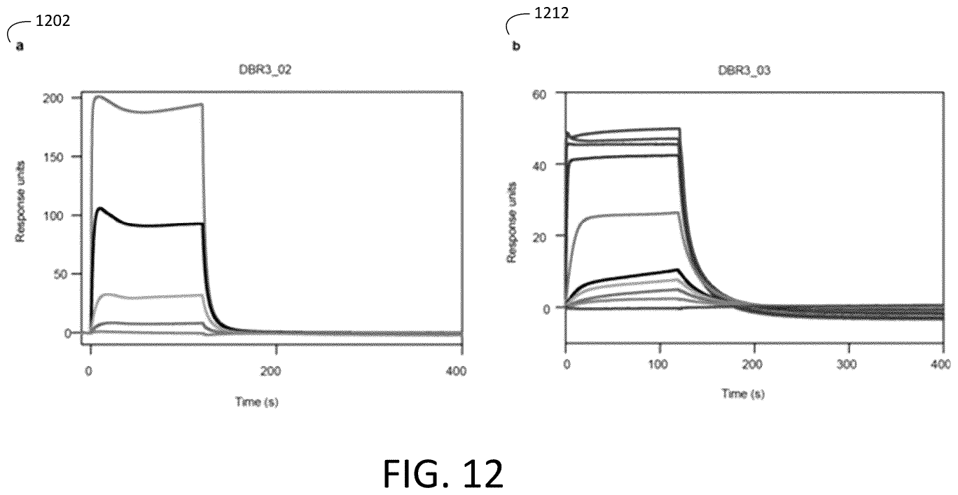

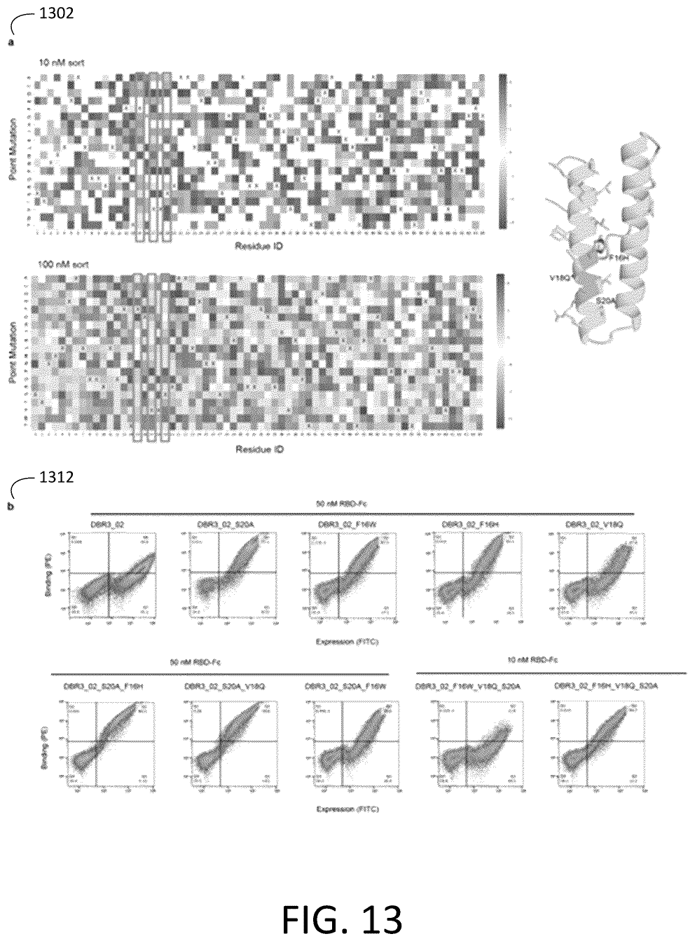

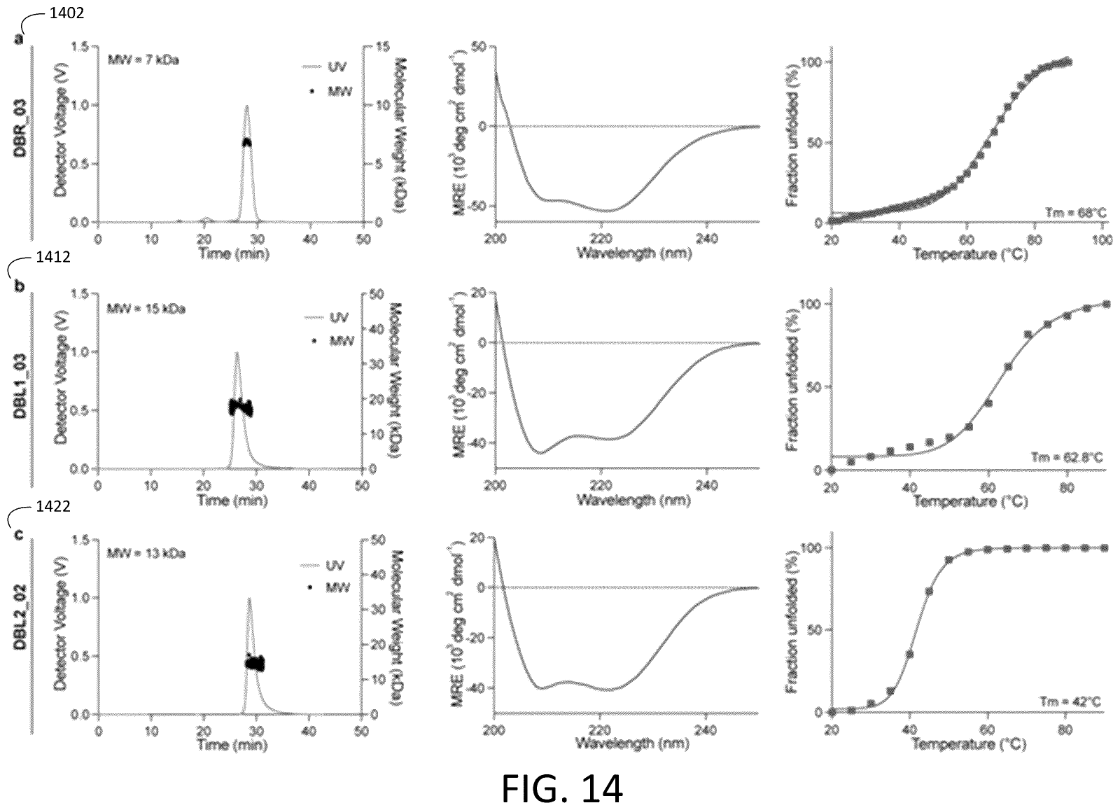

Next, using the Rosetta MotifGraft protocol we identified several protein scaffolds compatible with both binding modes of the seed (FIG. 2c), transplanted the seed hotspot side chains from a top-ranking seed, and designed the remaining residues of the interface (FIG. 1c). Sixty-three designs based on twenty scaffolds, ranging from 7 to 23 mutations relative to the native proteins, were screened with yeast display (FIG. 10a-c). From this initial round of designs, DBR3_01 showed weak binding in yeast display experiments that was competed by soluble ACE2 (FIG. 10d), suggesting that the binder was targeting the correct RBD site. Furthermore, DBR3_01 showed increased binding compared to the WT scaffold protein and a double point mutant on the designed interface residues, further supporting that the seed residues were participating in the binding interaction (FIG. 10e). Next, we sought to improve the binding affinity of the design by performing two mutagenesis libraries: first, a directed library (FIG. 11) in the design interface which yielded DBR3_02 with 4 mutations and a K_{D} of >4 μM determined by Surface Plasmon Resonance (SPR) (FIG. 2d, FIGS. 11,12); second, we screened a site saturation mutagenesis (SSM) library which resulted in the enrichment of 3 point mutants (FIG. 13). Adding these 3 mutations to DBR3_02 resulted in DBR3_03 that showed a K_{D} of 80 nM determined by SPR and was folded and stable (FIG. 2d, FIG. 12b, FIG. 14a). We started from a computationally designed binder with very low affinity as observed with yeast display, yet undetectable by SPR, and upon 6 mutations we observed an improvement greater than 60 fold in binding affinity. The mutations all occurred in the binding helix of the design. A17G and S20A in the core of the interface appear to have relieved steric clash and reduced buried unsatisfied polar atoms, respectively. Other mutations optimize the interface rim, increasing affinity.

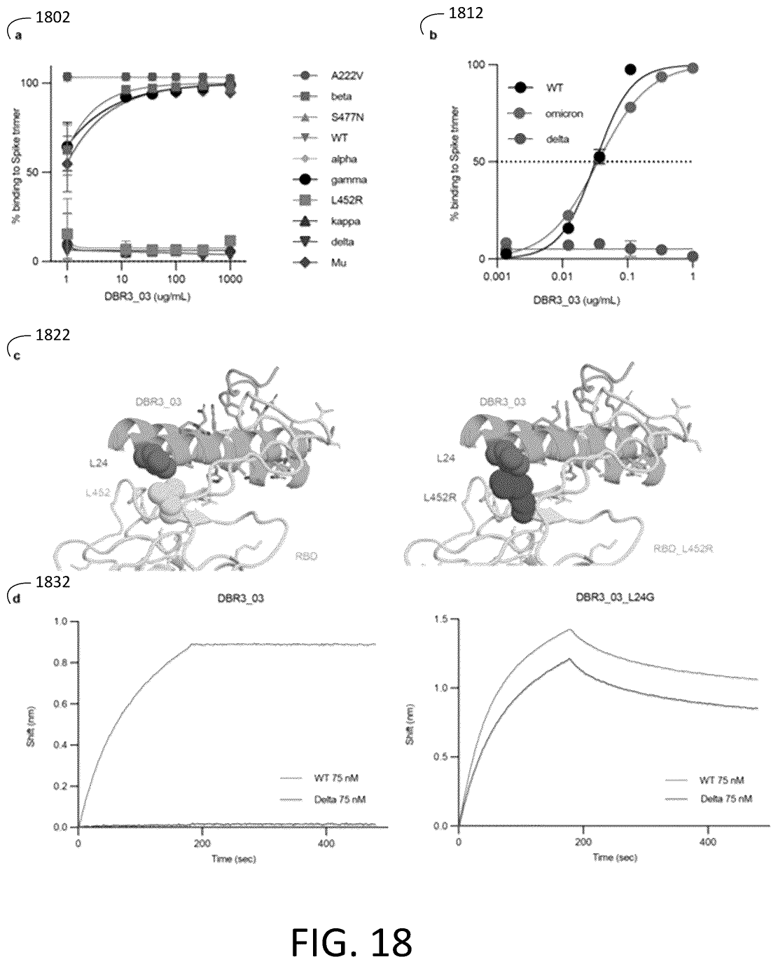

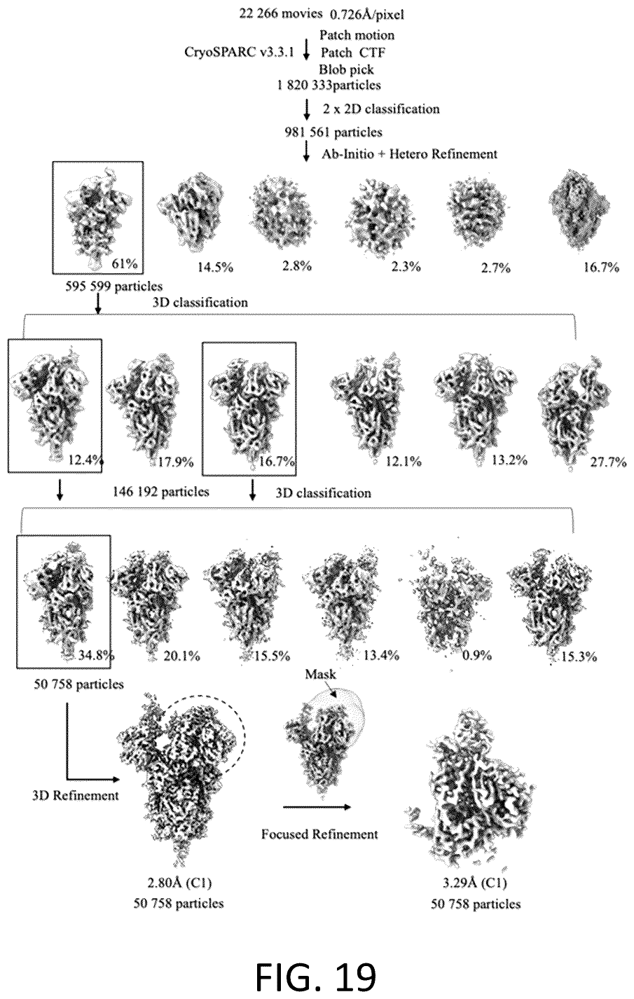

To structurally characterize the binding mode of DBR3_03 we solved a cryo-EM structure of the design in complex with the trimeric spike protein at 2.9 Å local resolution (FIG. 2e and FIGS. 15-17). The structure confirmed the predicted binding sites on both partners. Importantly, the binder adopted the orientation of the helical binding motif that was marginally less favored by MaSIF's fingerprint descriptors (down orientation) upon docking of the full protein (FIG. 2b,c). Interestingly, the initial design DBR3_01 showed similar metrics when the interfaces were analyzed in both directions (FIG. 9b), pointing to known limitations of surface fingerprints in unbound docking type of problems. This led us to attempt another state of the art protein docking method, AlphaFold (AF) multimer to predict the complex of DBR3_03 with the spike RBD and obtained a 1.4 Å iRMSD between the AF prediction relative to the experimental structure (FIG. 2e). This result presents a powerful demonstration of the synergies between machine learning techniques purely based on structural features and those that leveraged large sequence-structure datasets for structure prediction tasks. At the structural level DBR3_03 engages the RBD with a 1452 Å² of buried interface area (surface area buried on both sides of the complex), which is much smaller than the average buried surface area of antibodies (approximately 2071±456 Å^{2 22}), yet still results in a high affinity interaction. The designed interface lacks canonical hotspot residues and engages the RBD through small residues and is composed of 21% backbone and 79% side chain contacts. Given the pandemic situation with SARS-CoV-2 and the general need for rational design of protein-based therapeutics to fight viral infections, we next engineered an Fc-fused DBR3_03 (Fc-DBR3_03) construct and tested its neutralization capacity on a panel of SARS-CoV-2 variants in virus-free and pseudovirus surrogate assays (FIGS. 2f,g, FIG. 18a,b). We compared the breadth and potency of our design to those of clinically approved monoclonal antibodies. In virus-free assays we observed that Fc-DBR3_03 had comparable potency to that of RGN87 for the WT spike and bound to the omicron strain while RGN87 did not (FIG. 2f). Neutralization activity in pseudovirus assays was tested and it was found that Fc-DBR3_03 neutralized omicron, albeit less potently than the AstraZeneca (AZN) clinically approved antibody mix (FIG. 2g). A cryo-EM structure showed that the binding mode was nearly identical (1.4 Å backbone RMSD) between DBR3_03-WT-RBD complex and DBR3_03-omicron-RBD complex (FIGS. 19-21). Importantly, Fc-DBR3_03 showed a very broad reactivity to many SARS-CoV-2 variants (FIG. 18a) which is due to the sequence conservation of the targeted site and the small binding footprint of the design. The design was sensitive to the L452R mutation present in the delta variant (FIG. 18b, FIG. 2f), but introducing a single point mutation (L24G) to relieve the clash between L452R and the binder led to the design binding to delta (FIG. 18c,d). Our results highlight the value of the surface fingerprinting approach to reveal target sites in viral proteins and for the subsequent design of functional antivirals with broad activity.

FIG. 2 illustrates an example design and optimization of a SARS-CoV-2 RBD binder and related systems and methods in accordance with various embodiments disclosed herein. As shown for FIG. 2, procedure or step 202 (section a) is executed or otherwise implemented (e.g., by one or more processors), where as shown for FIG. 2, a MaSIF-site prediction of interface propensity of the RBD is implemented. At step 202, the ACE2 binding footprint (yellow outline) is distinct from the predicted binding site (red).

Further as shown for FIG. 2, procedure or step 212 (section b) is executed or otherwise implemented (e.g., by one or more processors), where as shown for FIG. 2, the MaSIF-seed is used to predict helical seeds that cluster into anti-parallel orientations, in either up or down position(s). At step 212, sequence logo plots are used to highlight the similarity between the sequences of the two seed clusters, regardless of orientation.

Further as shown for FIG. 2, procedure or step 222 (section c) is executed or otherwise implemented (e.g., by one or more processors), where as shown for FIG. 2, a scaffold (PDB ID: 5vny) is used to make DBR3_01 allows for binding in up or down position, sharing similar footprints.

Further as shown for FIG. 2, procedure or step 232 (section d) is executed or otherwise implemented (e.g., by one or more processors), where as shown for FIG. 2, SPR data is demonstrated showing improved DBR3 binders with controls. In the example, DBR3_03 has an affinity of 80 nM with RBD.

Further as shown for FIG. 2, procedure or step 242 (section e) is executed or otherwise implemented (e.g., by one or more processors), where as shown for FIG. 2, a Cryo-EM structure (dark green) is aligned to the AlphaFold prediction with iRMSD=1.4 Å. The trimeric spike protein (gray) has one DBR3_03 bound per RBD (orange, pink, green).

Further as shown for FIG. 2, procedure or step 252 (section f) is executed or otherwise implemented (e.g., by one or more processors), where as shown for FIG. 2, Fc-DBR3_03 binds to WT spike and omicron spike, but not the delta spike. Fc-DBR3_03 has an EC50 of 3.2e-8 g/mL with WT and 3.5e-8 g/mL with omicron. Imdevimab (RGN87) has an EC50 of 8.2e-8 g/mL with WT and 1.7e-7 g/mL with delta.

Further as shown for FIG. 2, procedure or step 262 (section g) is executed or otherwise implemented (e.g., by one or more processors), where as shown for FIG. 2 Fc-DBR3_03 neutralizes live omicron-virus in cell-based inhibition assays with an EC50 of 1.7E-6 g/mL, compared to the AstraZeneca (AZD, 8895 and 1061) mix that has an EC50 of 2.9e-7 g/mL.

Targeting Flat Surface Sites in Immune Checkpoint Receptors.

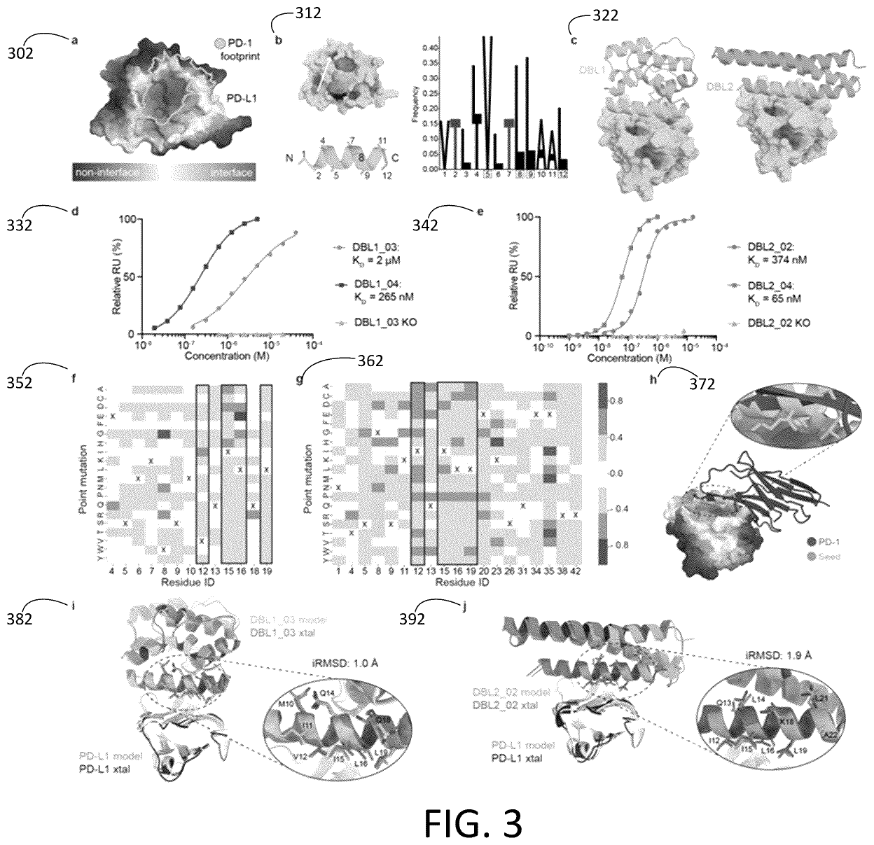

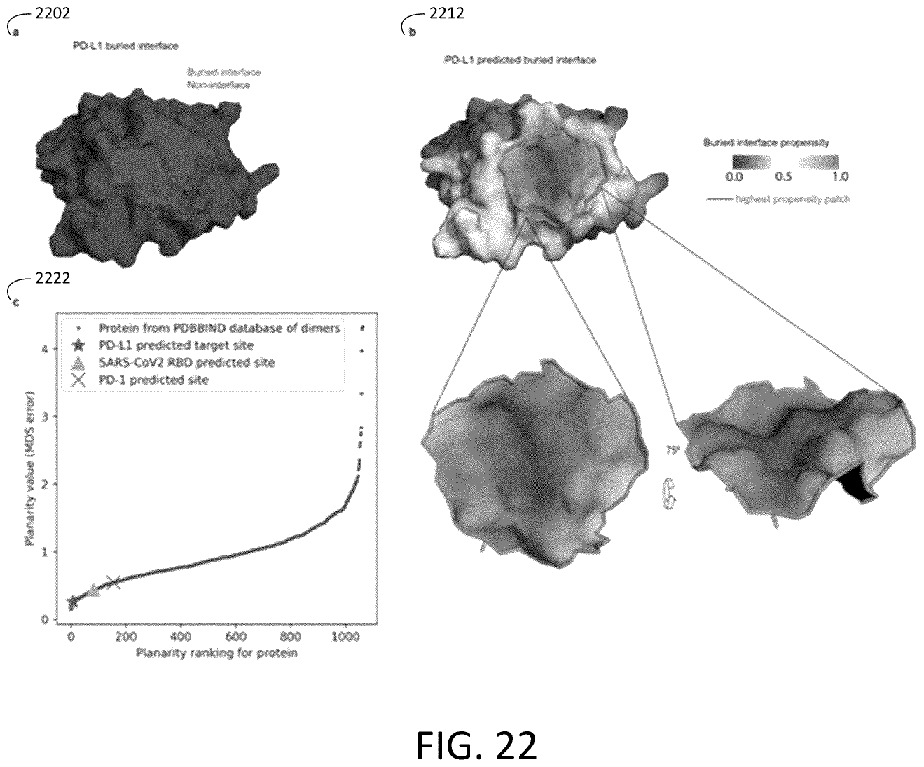

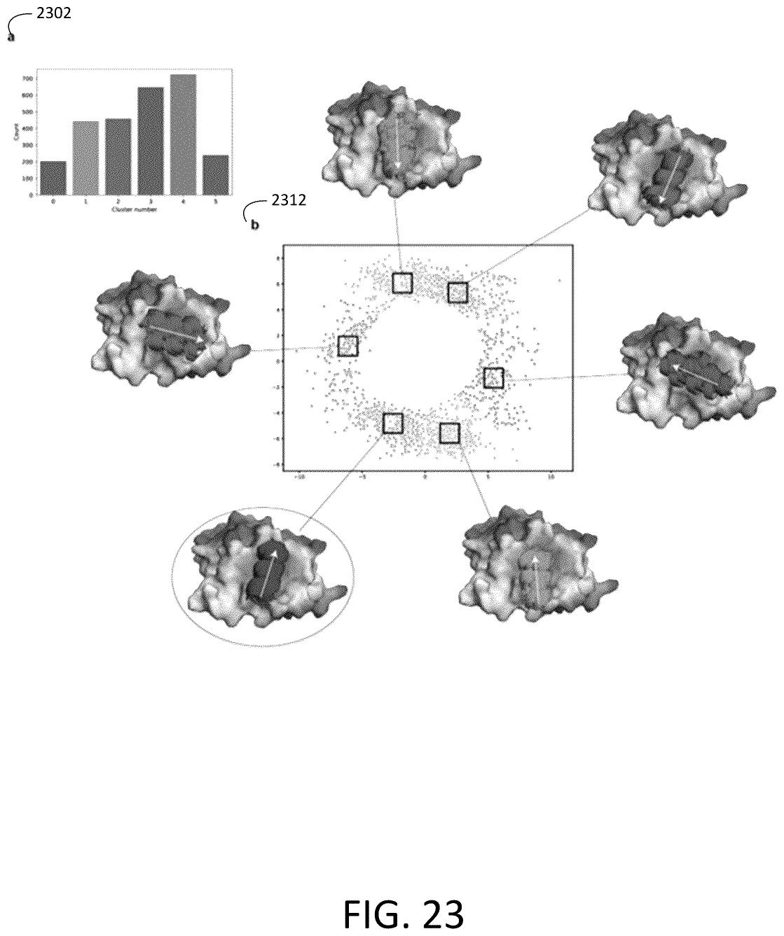

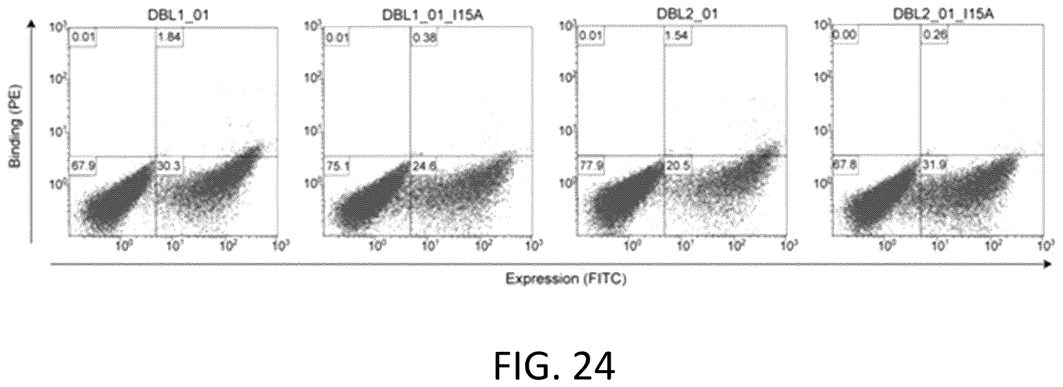

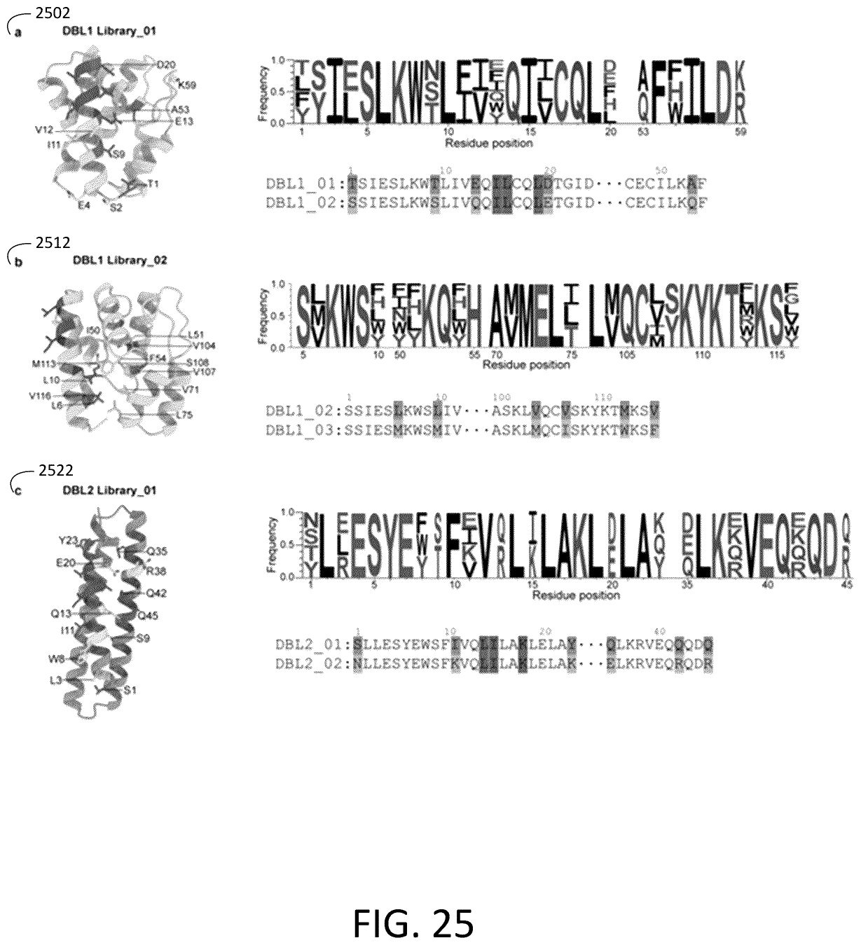

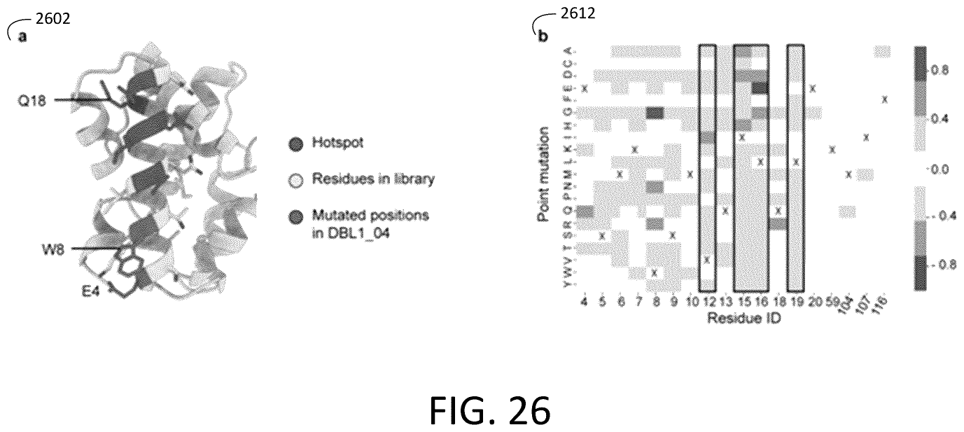

Surface sites presenting flat structural features are difficult to target with small molecule drugs, leading to their categorization as undruggable. To test our fingerprint-based approach, we sought to design binders to target the PD-1/PD-L1 interaction, which is central to the regulation of T-cell activity in the immune system. We used MaSIF-site to find high propensity protein binding sites in PD-L1, and unsurprisingly, the identified site overlapped significantly with the native binding site engaged by PD-1 (FIG. 3a). This site is extremely flat at the structural level, ranking in the 99^{th} percentile in terms of interface flatness (ranked #7 among 1068 transient interfaces) (FIG. 22). Next, we used MaSIF-seed to find binding motifs to engage the site, among the top results helical motifs clustered in both orientations packing in the beta-sheets of PD-L1 (FIG. 23b). In the most populated cluster (FIG. 23a), we observed sequence convergence for a 12 residue fragment (FIG. 3b). We then used Rosetta MotifGraft to search for scaffolds to display this fragment and used RosettaDesign to optimize contacts at the interface. We tested 16 designs based on 5 different scaffolds for binding to PD-L1 on the surface of yeast. Two designs based on two different scaffolds showed low binding signals (FIG. 24), which we refer to as DBL1_01 and DBL2_01 (FIG. 3c). The specificity of the interaction was confirmed by testing hotspot knockout controls of each design (FIG. 24). We performed mutational studies to improve DBL1_01's binding affinity and protein stability, for the former, a combinatorial library was constructed with mutations in the predicted binding region, while maintaining the hotspot residues predicted by MaSIF-seed (FIG. 25a). From this library we selected a variant, DBL1_02 with 5 mutations that were mostly mild changes in the interface rim of the design improving the formation of polar contacts. The most substantial change occurred at position 53, the mutation of alanine to glutamine allows for the formation of an additional hydrogen bond with PD-L1. (FIG. 25a). To improve the design's expression and stability we constructed a library targeting core residues to optimize core packing (FIG. 25b). Combining mutations from both libraries, we obtained DBL1_03 with 11 mutations from the starting design, which was folded and monomeric in solution and showed a binding affinity of 2 μM (FIG. 3d, FIG. 14b), comparable to that of PD-1 (K_{D}=8.2 μM). To further assess the optimality of each residue at the interface of the designed binder we screened an SSM library sampling 19 positions, based on DBL1_03. The most relevant positions are shown in FIG. 3f (all positions in FIG. 26a,b). The SSM results revealed that the four hotspot residues placed by MaSIF-seed were crucial, showing that generally any other residue was deleterious for binding (FIG. 3f). However, in the interface rim many mutations could provide affinity improvements strongly suggesting that this region of the interface was suboptimal (FIG. 3f). Based on these data, we generated the DBL1_04 variant which resulted in a 10-fold increase of the binding affinity showing a K_{D} of 256 nM to PD-L1 (FIG. 3d).

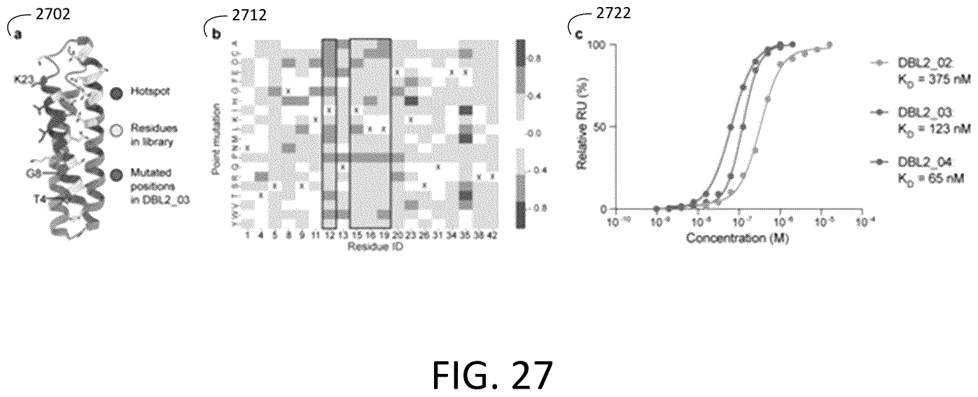

The second lead design based on a different scaffold, DBL2_01, could not be solubly expressed and therefore we designed a combinatorial library to improve expression and binding affinity (FIG. 25c). From this library we isolated the variant DBL2_02 which had six mutations and could be expressed in E. coli. From the six mutations, three were predicted to be in the interface (Y23K, Q35E, Q42R) and improved binding affinity by forming additional salt bridges with PD-L1 (FIG. 25c). The K_{D} to PD-L1 determined by SPR was 374 nM, more than 10-fold higher than the native ligand PD-1. Since both designs shared the same binding seed we transplanted the SSM mutations of the DBL1_04 design and generated the DBL2_03, which showed a 3-fold improvement in binding affinity (K_{D}=120 nM) (FIG. 27c), indicating that the binding seed was engaging PD-L1 in a similar fashion to that of DBL1_03. To further assess the influence of each residue in the designed binding interface we performed an SSM analysis on 19 interface residues of DBL2_03 (FIG. 3f, FIG. 27a,b). The SSM profile reiterated that the hotspot residues placed by MaSIF-seed were very restricted in variability, showing that these residues were accurately predicted. In contrast, several positions on the interface rim were suboptimal and mutations to polar amino acids resulted in affinity enhancements.

Based on the SSM data, we generated the DBL2_04 design with additional polar mutations (FIG. 3g, FIG. 27a) which showed an improved K_{D} of 65 nM (FIG. 3e). To experimentally validate the binding mode, we co-crystallized the designs with PD-L1. Overall for both designs, the structures (FIG. 3i,j) showed excellent agreement with our computational models with 0.8 Å and 2.0 Å for the overall backbone RMSDs of the complexes relative to the predicted binding modes for DBL1_03 and DBL2_02, respectively. The full atom interface RMSD was 1.0 Å and 1.9 Å for DBL1_03 and DBL2_02, respectively, showing an exquisite accuracy of the predictions in the interface region. The buried interface area of the designs with PD-L1 was between 1424 Å² and 1438 Å², compared to 1648 Å² for the buried interface area of PD-1 (PDB ID: 4ZQK). The chemical composition of the designed interface is similar in both designs, ˜59% of the surface area is hydrophobic and the remaining area is hydrophilic for DBL1_03 and correspondingly for DBL2_02. These values are comparable to those of the PD-1/PD-L1 interaction (52% hydrophobic surface), showing that we have designed interfaces with similar chemical compositions of the native interaction using a distinct backbone conformation (FIG. 3h). The discovery of novel binding motifs by MaSIF-seed is striking when comparing the backbone motif used by the native PD-L1 binding partner, PD-1, and the designed binders. While the native PD-1 uses a beta-hairpin to engage the site, the designed binders do so through an alpha-helix motif, clearly showing that our approach has the capability to explore outside of the structural repertoire of native binding motifs. The general trend arising from the designed PD-L1 binders is that despite the accurate predictions of core residues in the interface, through mutagenesis studies, the designed polar interactions are suboptimal. To address these and other limitations of our computational approach, we performed additional computational design steps to improve the pipeline and tested it on the design of binders to target PD-1.

FIG. 3 illustrates an example design and optimization of PD-L1 binder designs and related systems and methods in accordance with various embodiments disclosed herein. As shown for FIG. 3 procedure or step 302 (section a) is executed or otherwise implemented (e.g., by one or more processors), where as shown for FIG. 3, a MaSIF-site prediction of interface propensity of PD-L1 may be implemented. At step 302, the predicted interface (red) overlaps with the binding site of the native interaction partner PD-1 (yellow).

Further as shown for FIG. 3, procedure or step 312 (section b) is executed or otherwise implemented (e.g., by one or more processors), where as shown for FIG. 3, helical seeds were predicted by MaSIF-seed and clustered. At step 312, the dominant cluster shows strong amino acid preferences (Z-score >2). Hotspot residues are framed in black boxes.

Further as shown for FIG. 3, procedure or step 322 (section c) is executed or otherwise implemented (e.g., by one or more processors), where as shown for FIG. 3, binders based on two different scaffold proteins utilizing the selected seed were identified.

Further as shown for FIG. 3, procedure or step 332 (section d) is executed or otherwise implemented (e.g., by one or more processors), where as shown for FIG. 3, binding affinities are shown for DBL1 designs after combinatorial (light green) and SSM library optimization (dark green), measured by SPR. At step 332, mutation of a hotspot residue (V12R) ablates binding of DBL1_03 (wheat).

Further as shown for FIG. 3, procedure or step 342 (section e) is executed or otherwise implemented (e.g., by one or more processors), where as shown for FIG. 3, binding affinities is demonstrated for DBL2 designs after combinatorial (light blue) and SSM library optimization (dark blue), measured by SPR. At step 342, mutation of a hotspot residue (V12R) knocks out binding of DBL2_02 (wheat).

Further as shown for FIG. 3, procedure or step 352 (section f) is executed or otherwise implemented (e.g., by one or more processors), where as shown for FIG. 3, SSM for regions of interest are shown for the binding interface of DBL1_03. At step 352, the “x” indicates the original residue of DBL1_03. Hotspot residue positions are framed in black boxes. Blue indicates enrichment in the binding population, while red shows enrichment in the non-binding population.

Further as shown for FIG. 3, procedure or step 362 (section g) is executed or otherwise implemented (e.g., by one or more processors), where as shown for FIG. 3, SSM data is demonstrated in the binding interface of DBL2_03. At step 362, the “x” indicates the original residue of DBL2_02.

Further as shown for FIG. 3, procedure or step 372 (section h) is executed or otherwise implemented (e.g., by one or more processors), where as shown for FIG. 3, the binding mode of the selected seed is demonstrated in comparison with the native interaction partner PD-1.

Further as shown for FIG. 3, procedure or step 382 (section i) is executed or otherwise implemented (e.g., by one or more processors), where as shown for FIG. 3, structural validation of DBL1_03 is demonstrated by aligning the computational model (lighter color) with the crystal structure (darker color). At step 382, the inset shows the alignment of the residues in the binding seed.

Further as shown for FIG. 3, procedure or step 392 (section j) is executed or otherwise implemented (e.g., by one or more processors), where as shown for FIG. 3, structural validation of DBL2_02 is demonstrated by aligning the computational model (light green) with the crystal structure (dark green). At step 392, the inset shows the alignment of the residues in the binding seed represented in sticks.

One-Shot Design De Novo Protein Binders with Native-Like Affinites.

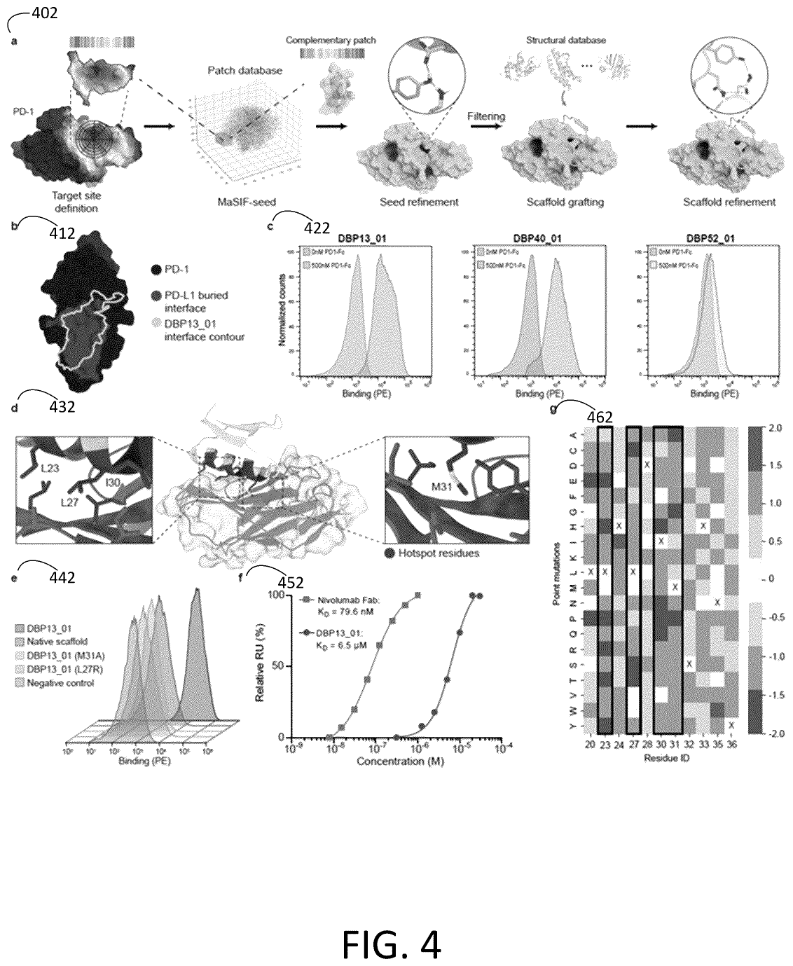

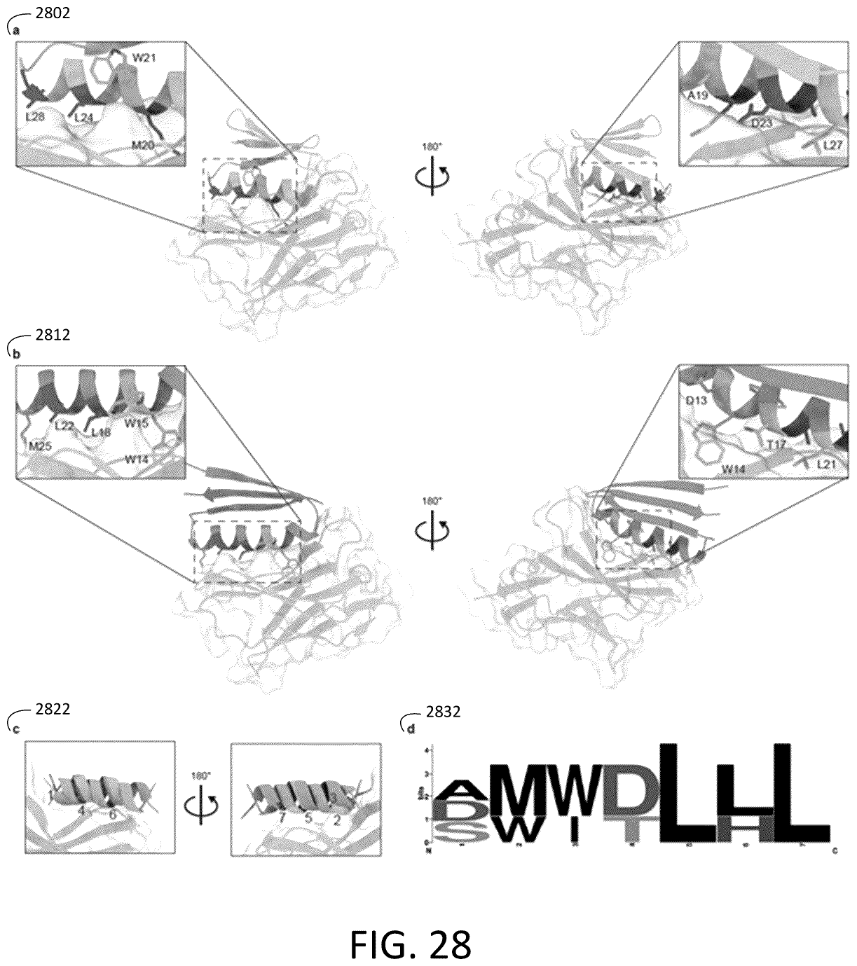

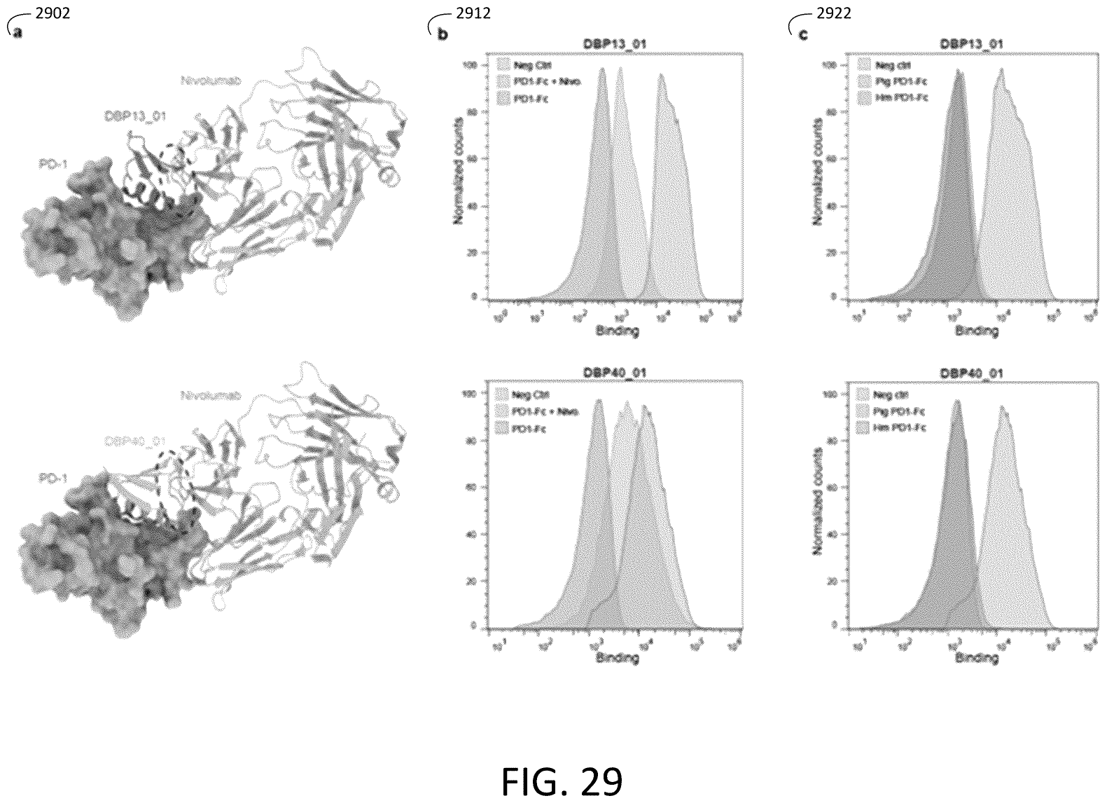

Despite the successes in designing site specific binders to engage two different targets, the computational designs still required in vitro evolution to enable expression and detectable binding affinities that could be biochemically characterized. To address these issues we assembled a more comprehensive design pipeline (FIG. 4a) performing: I) sequence optimization of selected seeds; and II) biased design for polar contacts in the scaffold interface. To test this approach, we designed de novo binders to target the PD-1 site that is natively engaged by PD-L1 (FIG. 4b). We tested the top 2000 designed sequences according to several structural metrics (see Methods) by yeast display. Using a deep sequencing readout (see methods), three designs based on de novo miniprotein scaffolds (DBP13_01, DBP40_01 and DBP52_01) showed a moderate to strong binding signal on the surface of yeast (FIG. 4c, FIG. 28). The most promising candidate, DBP13_01, was investigated in more detail (FIG. 4b,c,d). To confirm whether the binding interaction was mediated through the designed interface, we tested several control constructs, which included the native miniprotein scaffold and DBP13_01 variants with predicted knockout mutations (FIG. 4d), all of which abolished binding (FIG. 4e). The interaction site on PD-1 was further probed via a competition assay with Nivolumab, which blocked the DBP13_01/PD-1 interaction as expected due to the overlapping binding footprints (FIG. 29). The DBP13_01/PD-1 interaction showed a K_{D} of 6.5 μM (FIG. 4f) as determined by SPR, similar to the affinity of the native PD-L1/PD-1 interaction (K_{D}=8.2 μM).

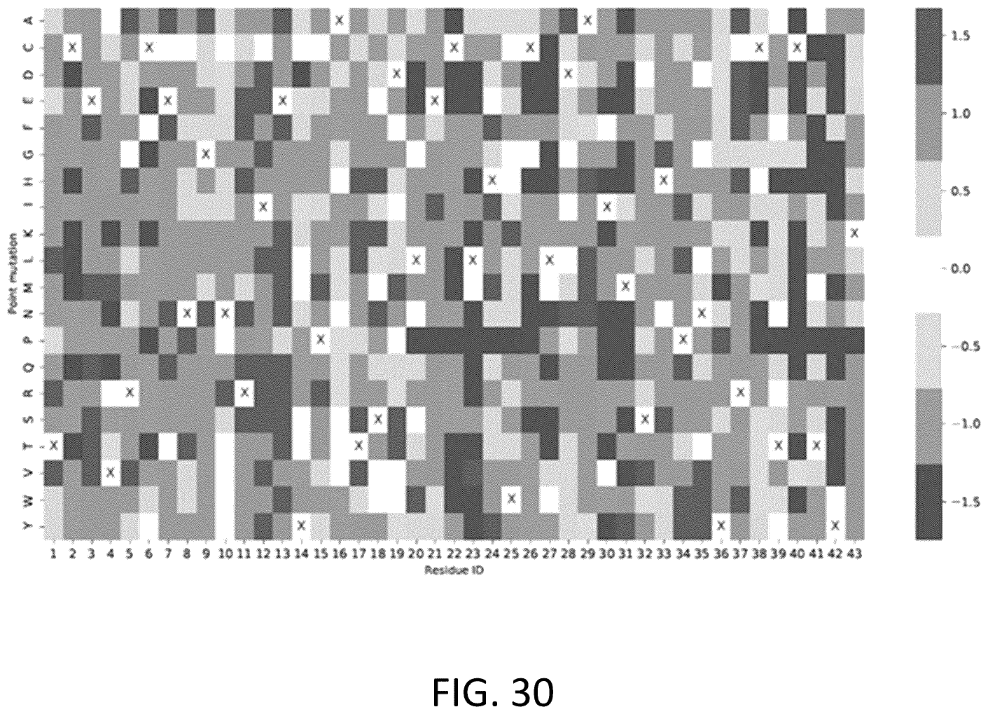

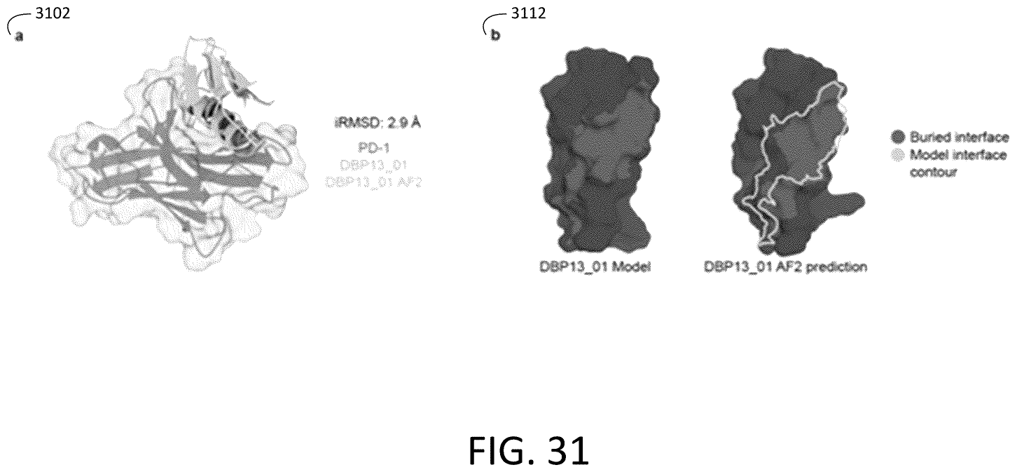

This was a promising result given that the design was not subjected to experimental optimization by in vitro evolution. Next, we performed an SSM study and we observed that mutations at the predicted core interface positions (L23, L27, 130, M31) were generally deleterious for binding, supporting the structural and sequence accuracy of the design (FIG. 4g, FIG. 30). The predicted complex structure by AlphaFold Multimer was in agreement with that of MaSIF, with an interface footprint that is largely overlapping with the designed residues, and 3.3 Å of backbone RMSD and 2.9 Å of interface full atom RMSD (FIG. 31). Although these results are supported by the SSM data, they are a predictive exercise and cannot be interpreted as the absolute evidence that the designed binding mode is occurring, which ultimately will require an experimental structure. Overall, the results show that by starting the interface design process driven by surface fingerprints and introducing additional features of native interfaces (e.g. hotspot optimization, polar contacts) we can design site-specific binders with native-like affinities purely by computational design.

FIG. 4 illustrates an example optimized workflow and de novo binders for PD-1 and related systems and methods in accordance with various embodiments disclosed herein. As shown for FIG. 4 procedure or step 402 (section a) is executed or otherwise implemented (e.g., by one or more processors), where as shown for FIG. 4, an improved design computational workflow is demonstrated where two steps of design are used, at the seed and at the scaffold level, with an emphasis on building new hydrogen bond networks.

Further as shown for FIG. 4, procedure or step 412 (section b) is executed or otherwise implemented (e.g., by one or more processors), where as shown for FIG. 4, a PD-1 surface (blue) with the region targeted by PD-L1 (red) is demonstrated with an overlapping region targeted by DBP13_01 (yellow contour).

Further as shown for FIG. 4, procedure or step 422 (section c) is executed or otherwise implemented (e.g., by one or more processors), where as shown for FIG. 4, histograms demonstrate the binding signal (PE) measured on three yeast clones displaying designed binders against PD-1. At step 422, yeasts are labeled with 500 nM PD1-Fc (coloured) or secondary antibodies only (Grey, negative control).

Further as shown for FIG. 4, procedure or step 432 (section d) is executed or otherwise implemented (e.g., by one or more processors), where as shown for FIG. 4, PD-1 structure (blue) is demonstrated targeted by DBP13_01 (green) with highlighted hotspots residue from the binding seed (red). At step 432, the two neighboring boxes indicate crucial residue for binding.

Further as shown for FIG. 4, procedure or step 442 (section e) is executed or otherwise implemented (e.g., by one or more processors), where as shown for FIG. 4, a histogram demonstrates the binding signal measured by flow cytometry for DBP13_01, the native miniprotein scaffold, two variants of DBP13_01 with crucial residues mutated, and a negative control with unlabeled yeast.

Further as shown for FIG. 4, procedure or step 452 (section f) is executed or otherwise implemented (e.g., by one or more processors), where as shown for FIG. 4, binding affinities are demonstrated as determined by SPR of Nivolumab Fab (green squares) and DBP13_01 (red circles).

Further as shown for FIG. 4, procedure or step 462 (section g) is executed or otherwise implemented (e.g., by one or more processors), where as shown for FIG. 4, SSM heatmap show interface residues and the enrichment of each point mutation. At step 462, the “x” indicates the original amino acid identity in DBP13_01. Blue indicates enrichment in the binding population, while red shows enrichment in the non-binding population. Hotspot residues are highlighted with a black box.

Physical interactions between proteins in living cells are one of the hallmarks of function. Our incomplete understanding of the complex interplay of molecular forces that drive PPIs has greatly hindered the comprehension of fundamental biological processes as well as the capability to engineer such interactions from first principles. It has been particularly challenging for protein modeling methodologies that use discrete atomic representations to perform de novo design of PPIs. In large part this is due to the small number of molecular interactions involved in most protein interfaces and to the very small energetic contributions that determine binding affinities, making physics-based energy functions less reliable. To address this gap we developed an enhanced data framework to represent proteins as surfaces and learn the geometric and chemical patterns that ultimately determine the propensity of two molecules to interact. We proposed a new geometric deep learning tool, MaSIF-seed, to overcome the PPI design challenge by both identifying patches with a high propensity to form buried surfaces and binding seeds with complementary surfaces to those patches. By embedding molecular surfaces as numerical fingerprints, we rapidly and reliably identify complementary surface fragments that can engage a specific target within 140 million candidate surfaces, solving an important challenge in protein design by efficiently handling search spaces of daunting scales.

The identified binding seeds were then used as the interface driving core to design novel binding proteins against three challenging targets: a novel predicted interface in the SARS-CoV-2 spike protein, which ultimately yielded a SARS-CoV-2 inhibitor and the PD-1/PD-L1 protein complex, which exemplifies sites that are difficult to target with small molecules due to its flat surface. Multiple designed binders showed close mimicry to computationally predicted models and achieved high binding affinities, often, after experimental optimization. In the case of the PD-1 binder, the design showed low micromolar affinity without experimental optimization, which is the range of many native PPIs. By using surface fingerprints we identified novel structural motifs that can mediate de novo PPIs presenting a route to expand the landscape of motifs that can be used to functionalize proteins and be critical for the de novo design of function.

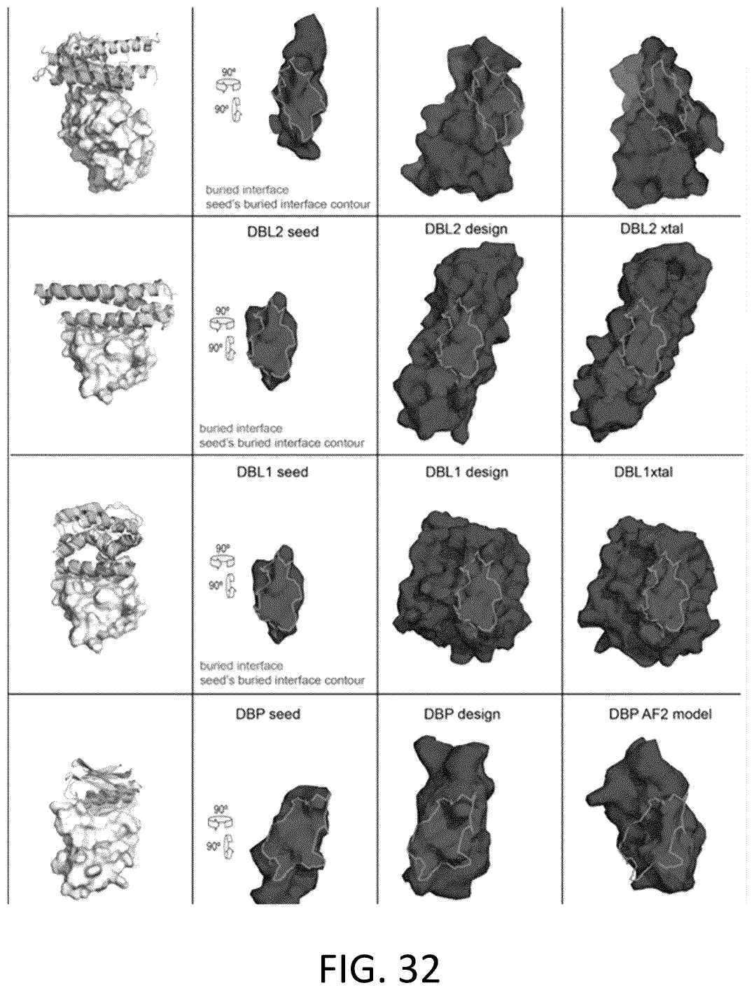

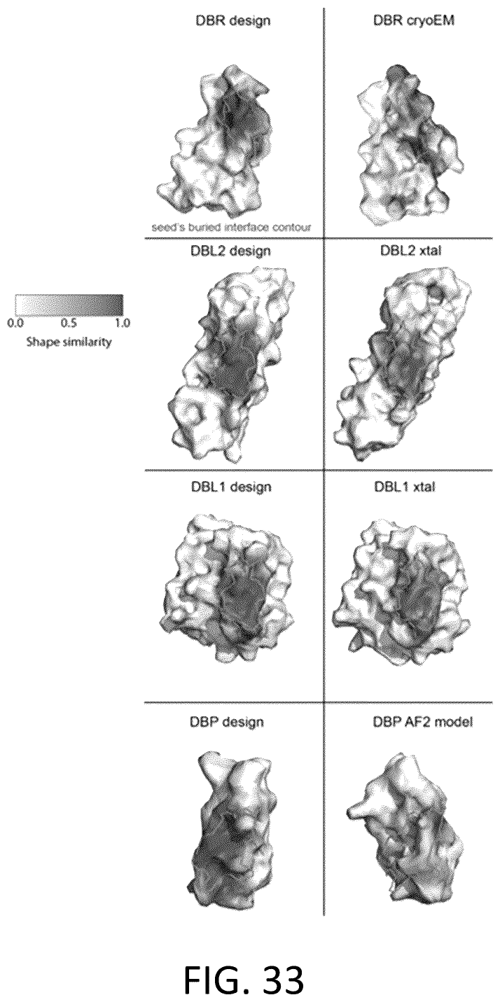

For all three targets, the original binding seed arguably provided the principal driver of molecular recognition representing the design's binding interface core (FIG. 32), maintaining a high surface similarity in this region (FIG. 33). However, contacts at the buried interface region are necessary but in most cases, likely not sufficient for high affinity binding, and in the three designed binders for PD-L1 and RBD, optimization of the polar interface rim through libraries was necessary to improve binding to a biochemically detectable range (K_{D} at the micromolar level). Our de novo designs highlight previous findings that small changes in the polar interface rim (for example, in the water network or hydrogen bond network surrounding the interface) can result in substantial differences in binding affinities. Encouragingly, the PD-1 binders designed using the optimized pipeline showed binding affinities in the low micromolar range without the need for experimental optimization, which represents a major step forward for the robust design of de novo PP Is.

In our study several limitations of the approach became evident, namely the absence of conformational flexibility and adaptation of the protein backbone to mutations and the difficulty of designing polar interactions that balance the hydrophobic patches of the interface contributing for affinity and specificity, which has also been observed by other authors. In future methodological developments, neural network architectures could be optimized to capture such features of native interfaces. Lastly, our approach is intrinsically limited by the restricted structural space used for binding seeds with helical structure, which present advantages in terms of the modularity and predictability of their structures, but are unlikely to be the universal solution for the de novo PPI design problem. Most likely, a larger diversity of structural motifs will be necessary and we anticipate the emergence of generative algorithms that can construct backbones conditioned to the target binding sites.

Here we presented a surface-centric design approach that leveraged molecular representations of protein structures based on learned geometrical and chemical features. We showed that these structural representations can be efficiently used for the design of de novo protein binders, one of the most challenging problems in computational protein design. We anticipate that this conceptual framework for generation of rich descriptors of molecular surfaces can open possibilities in other important biotechnological fields like drug design, biosensing or biomaterials in addition to providing a means to study interaction networks in biological processes at the systems levels.

FIG. 5 illustrates an example overview of the neural network architectures used in the MaSIF protocols and related systems and methods in accordance with various embodiments disclosed herein. As shown for FIG. 5 procedure or step 502 (section a) is executed or otherwise implemented (e.g., by one or more processors), where as shown for FIG. 5, a general MaSIF framework is demonstrated.

Further as shown for FIG. 5, procedure or step 512 (section b) is executed or otherwise implemented (e.g., by one or more processors), where as shown for FIG. 5, an MaSIF-site neural network is demonstrated.

Further as shown for FIG. 5, procedure or step 522 (section c) is executed or otherwise implemented (e.g., by one or more processors), where as shown for FIG. 5, an MaSIF-search neural network is demonstrated.

Further as shown for FIG. 5, procedure or step 532 (section d) is executed or otherwise implemented (e.g., by one or more processors), where as shown for FIG. 5, the interface post-alignment scoring neural network is demonstrated.

FIG. 6 illustrates an example modeling buried surfaces as radial patches and related systems and methods in accordance with various embodiments disclosed herein. As shown for FIG. 6 procedure or step 602 (section a) is executed or otherwise implemented (e.g., by one or more processors), where as shown for FIG. 6, a histogram of the patch areas of thousands of randomly selected protein patches with a fixed radius of 12 Å is demonstrated.

Further as shown for FIG. 6, procedure or step 612 (section b) is executed or otherwise implemented (e.g., by one or more processors), where as shown for FIG. 6, a histogram of the area of the buried surface area on 1380 dimeric PPIs is demonstrated. At step 612, we note that areas are computed for only one of the proteins (i.e. each subunit in a PPI is computed separately), and that we used the solvent excluded surface area, while other authors report buried areas on the solvent accessible area that include the buried surface area of both proteins.

Further as shown for FIG. 6, procedure or step 622 (section c) is executed or otherwise implemented (e.g., by one or more processors), where as shown for FIG. 6, a size of the maximum inscribed radial patch for the 1380 proteins is demonstrated. At step 622, the patch area for the patches used here is 12 Å, for a set of 30,000 randomly selected patches.

Further as shown for FIG. 6, procedure or step 632 (section d) is executed or otherwise implemented (e.g., by one or more processors), where as shown for FIG. 6, an example of the buried interface area for two well known, high affinity binders, Immunity Protein IM9 (PDB ID: 1 EMV) and the protein Barnase (PDB ID: 1BRS) is demonstrated. At step 632, the buried interface of each protein when bound to its partner is shown in red. The maximum inscribed radial patch's circumference is shown in black, and the circumference of a patch with radius 12 Å is shown in green.

Further as shown for FIG. 6, procedure or step 642 (section e) is executed or otherwise implemented (e.g., by one or more processors), where as shown for FIG. 6, a histogram of similarities between MaSIF-search fingerprint similarity between: (blue) pairs of patches that are co-crystallized from transient PPI s, with the fingerprint computed for the patch centered on the largest inscribed radial patch, and (orange) pairs of patches where one was taken from the center of the interface of a random PPI and the other was taken from a random patch surface.

FIG. 7 illustrates an example benchmarking the identification of helices based on patches and related systems and methods in accordance with various embodiments disclosed herein.

As shown for FIG. 7, a non-redundant set of all protein chains in the Protein Data Bank was decomposed into continuous segments of helices, resulting in over 250K helices. The surfaces of all helices were computed and decomposed into radial patches, and for each patch a fingerprint was computed, resulting in 140M patches. In parallel, a benchmark test set was built with 27 receptors that bind helical peptides. The helical peptides were inserted into the databases of helices and decomposed into patches as well. Then, a buried interface was predicted in the receptor and a fingerprint was computed. Finally, our method was applied to find the most complementary patch for the predicted buried interface. The table in the bottom shows the number of recovered complexes for the 27 members of the benchmark, and how many were recovered in the short list of the top 10, top 100, top 1000 and which ones failed. The total time for all receptors is shown in the right.

FIG. 8 illustrates an example MaSIF-site target site prediction on SARS-CoV-2 RBD, PD-L1, and PD-1 and related systems and methods in accordance with various embodiments disclosed herein. As shown for FIG. 8, surface mode shows a per-surface-vertex regression score on the propensity of each point on the surface to form an interface. ranging from 0 (blue) to 1 (red) a-c, Predictions on each target, with the natural ligand of the target shown in cartoon as a reference. Above shows a view highlighting the predicted site, while below a 180 degree rotation is shown. As shown for FIG. 8 procedure or step 802 (section a) is executed or otherwise implemented (e.g., by one or more processors), where as shown for FIG. 8, the MaSIF-site prediction on SARS-CoV-2 RBD (PDB ID: 6M17), with the RBD shown in surface and the ACE2 in beige is demonstrated.

Further as shown for FIG. 8, procedure or step 812 (section b) is executed or otherwise implemented (e.g., by one or more processors), where as shown for FIG. 8, a prediction on PD-L1 (PDB ID: 5JDS), with PD-1 shown in purple is demonstrated.

Further as shown for FIG. 8, procedure or step 822 (section c) is executed or otherwise implemented (e.g., by one or more processors), where as shown for FIG. 8, a prediction on PD-1 (PDB ID: 4ZQK) with the natural binder PD-L1 shown in cyan is demonstrated.

FIG. 9 illustrates an example RBD-binder metrics for up- and down-orientation and related systems and methods in accordance with various embodiments disclosed herein. As shown for FIG. 9 procedure or step 902 (section a) is executed or otherwise implemented (e.g., by one or more processors), where as shown for FIG. 9, a distribution of the IPA scores for the seeds of the up and down orientation and respective cluster sizes is demonstrated.

Further as shown for FIG. 9, procedure or step 912 (section b) is executed or otherwise implemented (e.g., by one or more processors), where as shown for FIG. 9, an interface metrics of DBR3_01 for the up- and down-orientation is demonstrated.

FIG. 10 illustrates an example original RBD-binder designs displayed on yeast and related systems and methods in accordance with various embodiments disclosed herein. As shown for FIG. 10 procedure or step 1002 (section a) is executed or otherwise implemented (e.g., by one or more processors), where as shown for FIG. 10, pools of approximately 30 designs were displayed on the surface of yeast and the highest binding populations sorted for further analysis are demonstrated.

Further as shown for FIG. 10, procedure or step 1012 (section b) is executed or otherwise implemented (e.g., by one or more processors), where as shown for FIG. 10, a schematic of RBD (tan) bound to the various members of the library (pale silhouettes and purple for DBR3_01) and ACE2 (red) overlapping with the designed binders is demonstrated.

Further as shown for FIG. 10, procedure or step 1022 (section c) is executed or otherwise implemented (e.g., by one or more processors), where as shown for FIG. 10, individual designs DBR1-DBR20 are demonstrated.

Further as shown for FIG. 10, procedure or step 1032 (section d) is executed or otherwise implemented (e.g., by one or more processors), where as shown for FIG. 10, the DBR3_01 design displayed on yeast binds to RBD-Fc (left panel) but the binding is blocked when the RBD-Fc is preincubated with an excess of ACE2, indicating a competitive binding mode is demonstrated.

Further as shown for FIG. 10, procedure or step 1042 (section e) is executed or otherwise implemented (e.g., by one or more processors), where as shown for FIG. 10, the control constructs show less binding signal than the design, indicating that the design is engaging RBD with the predicted interface is demonstrated.

FIG. 11 illustrates an example directed library for DBR3_01 and related systems and methods in accordance with various embodiments disclosed herein. As shown for FIG. 11 procedure or step 1102 (section a) is executed or otherwise implemented (e.g., by one or more processors), where as shown for FIG. 11, a position of residues included in a combinatorial library to improve binding affinity is demonstrated.

Further as shown for FIG. 11, procedure or step 1112 (section b) is executed or otherwise implemented (e.g., by one or more processors), where as shown for FIG. 11, Sequence logo plot of specific mutations allowed within the library. The sequences list the residues mutated in DBR3_01 (highlighted in blue) and the mutations gained through the library in DBR3_02 (green) is demonstrated.

FIG. 12 illustrates an example SPR curves for DBR3 designs and related systems and methods in accordance with various embodiments disclosed herein. As shown for FIG. 12 procedure or step 1202 (section a) is executed or otherwise implemented (e.g., by one or more processors), where as shown for FIG. 12, SPR curves for DBR3_02 flowing over immobilized RBD. At step 1102, the highest concentration is 5 μM, decreasing by five-fold dilutions.

Further as shown for FIG. 12, procedure or step 1212 (section b) is executed or otherwise implemented (e.g., by one or more processors), where as shown for FIG. 12, SPR curves for DBR3_03 flowing over immobilized RBD are demonstrated. At step 1212, the highest concentration is 6.5 μM, decreasing by three-fold dilutions.

FIG. 13 illustrates an example SSM of DBR3_02 and related systems and methods in accordance with various embodiments disclosed herein. As shown for FIG. 13 procedure or step 1302 (section a) is executed or otherwise implemented (e.g., by one or more processors), where as shown for FIG. 13, heat maps of DBR3_02 SSM at two concentrations of RBD-Fc are demonstrated. At step 1302, X indicates the original amino acid of DBR3_02. Red indicates an enrichment of the mutation in the binding population, blue indicates an enrichment in the non-binding population. Three positions, green box, were enriched in both concentrations. The positions of these mutations are highlighted on the DBR3_03 structure.

Further as shown for FIG. 13, procedure or step 1312 (section b) is executed or otherwise implemented (e.g., by one or more processors), where as shown for FIG. 13, a yeast display of DBR3_02 with mutations from the SSM introduced shows increase in affinity to RBD is demonstrated.

FIG. 14 illustrates an example biophysical characterization of the designed binders and related systems and methods in accordance with various embodiments disclosed herein. As shown for FIG. 14 From left to right (for each of sections a-c 1402-1432), The oligomeric status was determined via multi-angled light scattering (MALS). Folding was measured using circular dichroism. Thermal stability was determined by plotting the ellipticity at 218 nm at increasing temperatures. The images in FIG. 14 correspond to the following: a, DBR3_03, b, DBL1_03, c, DBL2_02.

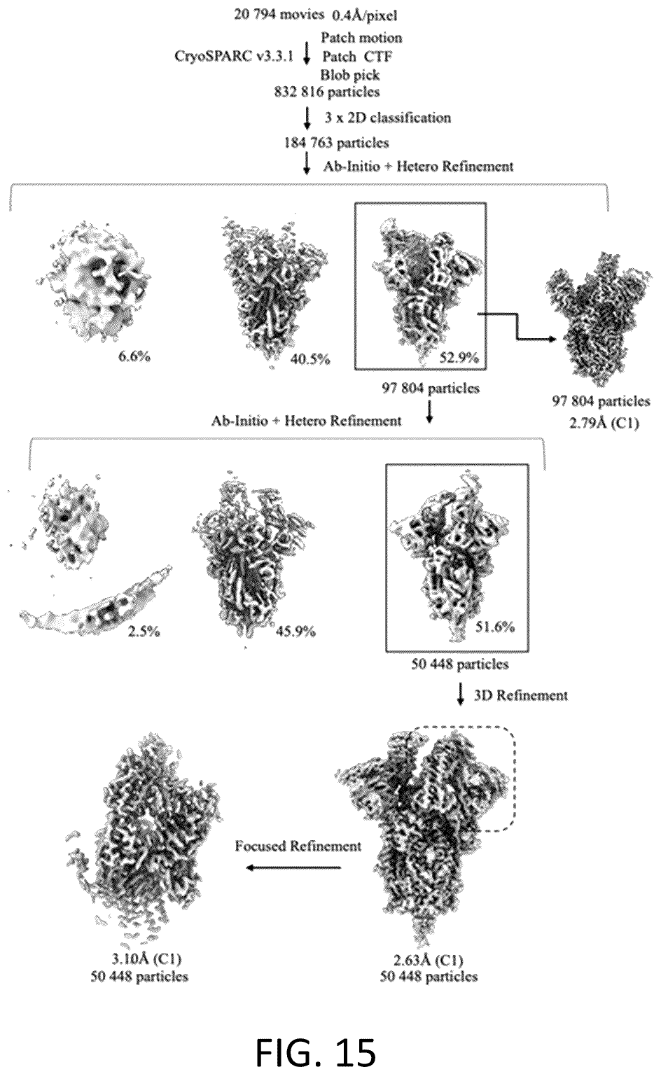

FIG. 15 illustrates an example cryo-EM structure of DBR3_03 and related systems and methods in accordance with various embodiments disclosed herein. As shown for FIG. 15, a structural characterization of the binding mode of DBR3_03 a cryo-EM structure of the design in complex with the trimeric spike protein at 2.9 Å local resolution is demonstrated.

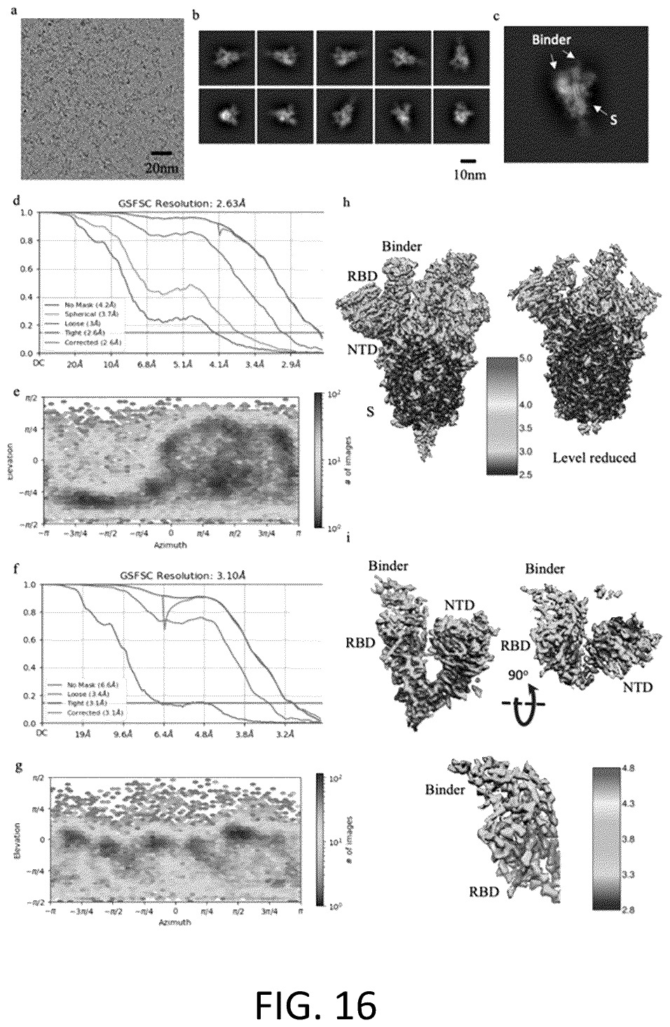

FIG. 16 illustrates an example GSFSC resolution mapping of RBD-binders and NTD-binders and related systems and methods in accordance with various embodiments disclosed herein. As shown for FIG. 16 (sections a-g), GSFSC resolution mappings of a cryo-EM structure of the design in complex with the trimeric spike protein at 2.9 Å local resolution are demonstrated.

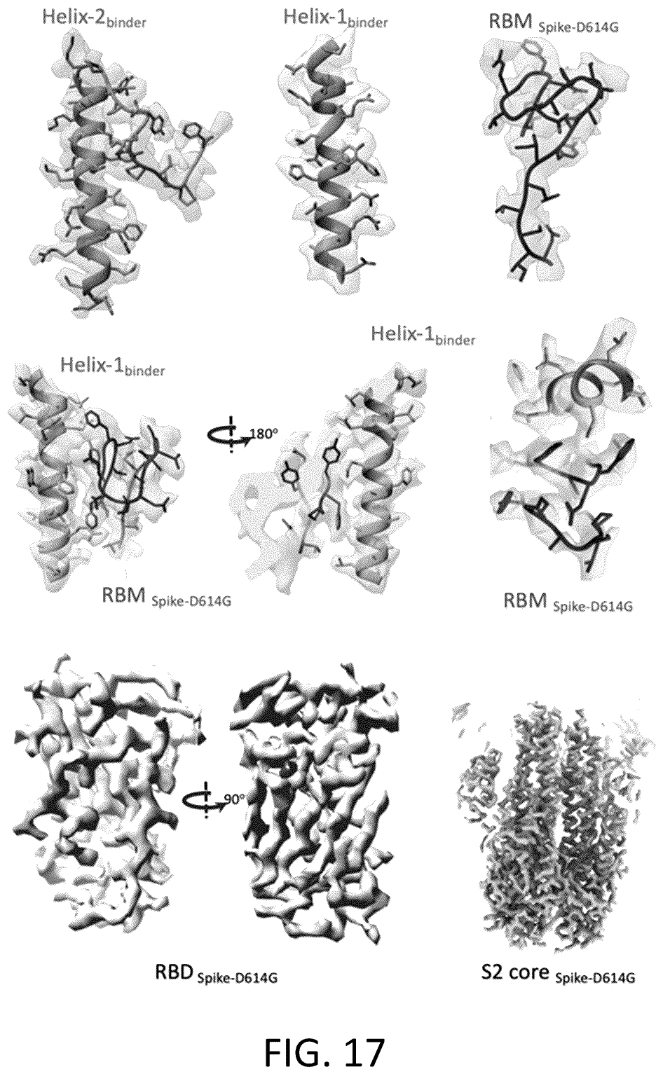

FIG. 17 illustrates an example spike-D614G and helix binders and related systems and methods in accordance with various embodiments disclosed herein. As shown for FIG. 17, spike-D614G and helix binders of a cryo-EM structure of the design in complex with the trimeric spike protein at 2.9 Å local resolution are demonstrated.

FIG. 18 illustrates an example DBR3_03 binds to several variants and related systems and methods in accordance with various embodiments disclosed herein. As shown for FIG. 18 procedure or step 1802 (section a) is executed or otherwise implemented (e.g., by one or more processors), where as shown for FIG. 18, a luminex binding assay of DBR3_03 with beads functionalized with SARS-CoV-2 spike protein of indicated variants is demonstrated.

Further as shown for FIG. 18, procedure or step 1812 (section b) is executed or otherwise implemented (e.g., by one or more processors), where as shown for FIG. 18, a luminex binding assay of DBR3_03 with the two main circulating strains (as of December 2021) and original variant is demonstrated.

Further as shown for FIG. 18, procedure or step 1822 (section c) is executed or otherwise implemented (e.g., by one or more processors), where as shown for FIG. 18, the L452R mutation on the spike protein leads to a clash with the DBR3_03 binding is demonstrated. At step 1822, the L24G mutation is proposed to avoid the clash.

Further as shown for FIG. 18, procedure or step 1832 (section d) is executed or otherwise implemented (e.g., by one or more processors), where as shown for FIG. 18, BLI data with DBR3_03 (WT KD<0.1 nM, delta KD not detected) or DBR3_03 L24G (delta KD=6 nM, WT KD=6 nM) immobilized on the tips, dipped into spike protein of different variants is demonstrated.

FIG. 19 illustrates an example cryo-EM structure of DBR3_03 and related systems and methods in accordance with various embodiments disclosed herein. As shown for FIG. 19, a structural characterization of the binding mode of DBR3_03 a cryo-EM structure of the design in complex with the trimeric spike protein at 2.9 Å local resolution is demonstrated. A cryo-EM structure showed that the binding mode was nearly identical (1.4 Å backbone RMSD) between DBR3_03-WT-RBD complex and DBR3_03-omicron-RBD complex.

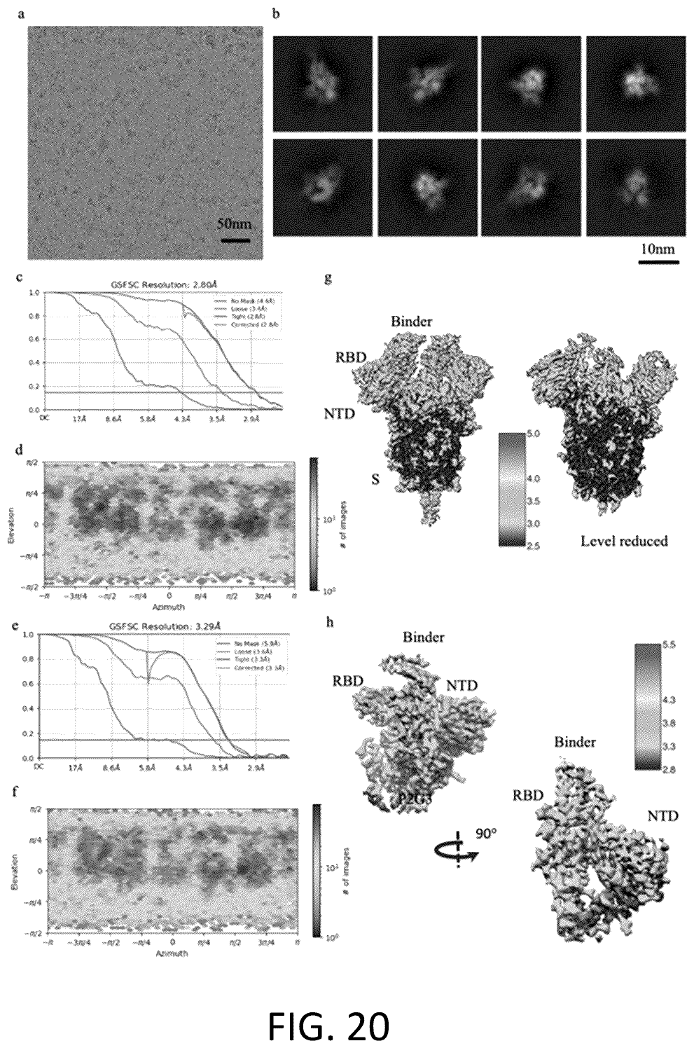

FIG. 20 illustrates an example GSFSC resolution mapping of RBD-binders and NTD-binders and related systems and methods in accordance with various embodiments disclosed herein. As shown for FIG. 20 (sections a-f), GSFSC resolution mappings of a cryo-EM structure of the design in complex with the trimeric spike protein at 2.9 Å local resolution are demonstrated.

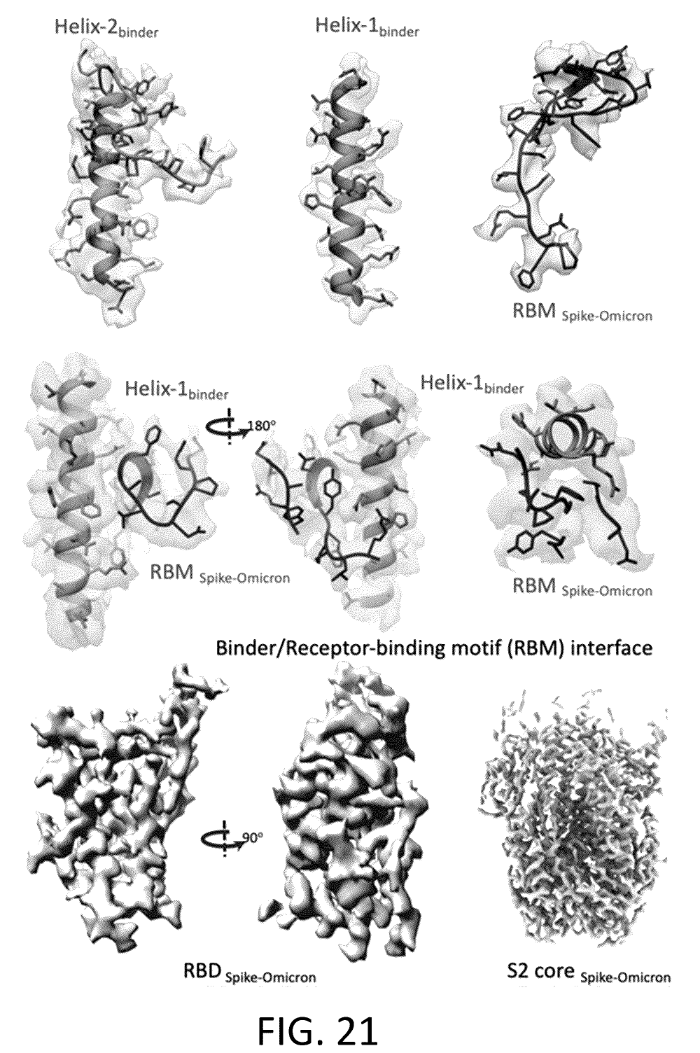

FIG. 21 illustrates an example spike-omicron and helix binders and related systems and methods in accordance with various embodiments disclosed herein. As shown for FIG. 17, spike-omicron and helix binders of a cryo-EM structure of the design in complex with the trimeric spike protein at 2.9 Å local resolution are demonstrated.

FIG. 22 illustrates an example planarity of the predicted targeted interfaces and related systems and methods in accordance with various embodiments disclosed herein. As shown for FIG. 22 procedure or step 2202 (section a) is executed or otherwise implemented (e.g., by one or more processors), where as shown for FIG. 22, a PD-L1 buried interface is demonstrated.

Further as shown for FIG. 22, procedure or step 2212 (section b) is executed or otherwise implemented (e.g., by one or more processors), where as shown for FIG. 22, a PD-L1 predicted buried interface, with selected target patch marked with a green contour is demonstrated above, and a view of the selected target patch to show its planarity is demonstrated below.

Further as shown for FIG. 22, procedure or step 2222 (section c) is executed or otherwise implemented (e.g., by one or more processors), where as shown for FIG. 22, a plotting of the planarity of each of 1068 dimeric protein interfaces is demonstrated. At step 2222, the Y-axis represents the error in multidimensional scaling when flattening the patch from 3D to 2D. The X-axis represents ranking of each protein according to the planarity value. The PD-L1 interface targeted in this work is marked with a red star, SARS-CoV-2 with a gold triangle, and PD-1 with a blue X.

FIG. 23 illustrates an example clusters of binding seeds docked on the PD-L1 surface and related systems and methods in accordance with various embodiments disclosed herein. As shown for FIG. 23, 140 million patches from 250,000 helices extracted from the PDB were compared and docked to the predicted interface in PD-L1 using MaSIF-seed-search. The top scoring seeds were selected for further processing. Twelve-amino acid fragments of these seeds that occupied the largest buried surface were then clustered using metric multi-dimensional scaling of all pairwise RMSDs between all seeds.

As shown for FIG. 23 procedure or step 2302 (section a) is executed or otherwise implemented (e.g., by one or more processors), where as shown for FIG. 23, a histogram of clusters, showing the prevalence of each orientation is demonstrated.

Further as shown for FIG. 23, procedure or step 2312 (section b) is executed or otherwise implemented (e.g., by one or more processors), where as shown for FIG. 23, a plot of the clusters in the multi-dimensional scaling plot is demonstrated. At step 2312, a box is drawn around the center of each cluster and the picture shows the selected helix orientation for all points inside the box. A star shows the location of the PD-L1 seed used for the designs.

FIG. 24 illustrates an example binding signals of initial PD-L1 binder designs and related systems and methods in accordance with various embodiments disclosed herein. As shown for FIG. 24, a binding measured on the surface of yeast using 15 μM PD-L1-Fc is demonstrated, showing a comparison of DBL1_01 and DBL2_01 with corresponding hotspot mutants.

FIG. 25 illustrates an example composition and outcome of yeast display libraries and related systems and methods in accordance with various embodiments disclosed herein. As shown for FIG. 25 procedure or step 2502 (section a) is executed or otherwise implemented (e.g., by one or more processors), where as shown for FIG. 25, a position of targeted residues in the structure of DBL1_01 to improve binding affinity is demonstrated. At step 2502, a logo plot of the allowed mutations in the library and alignment of initial design with library enriched design is further demonstrated.

Further as shown for FIG. 25, procedure or step 2512 (section b) is executed or otherwise implemented (e.g., by one or more processors), where as shown for FIG. 25, a position of targeted residues in the structure of DBL1_02 to improve core packing is demonstrated. At step 2512, a logo plot of the allowed mutations in the library and alignment of DBL1_02 with library enriched design is further demonstrated.

Further as shown for FIG. 25, procedure or step 2522 (section c) is executed or otherwise implemented (e.g., by one or more processors), where as shown for FIG. 25, a position of targeted residues in the structure of DBL2_01 to improve binding affinity and solubility is demonstrated. At step 2522, logo plot of the allowed mutations in the library and alignment of initial design with library enriched design is further demonstrated. Hotspot residues are shown red, targeted residues shown in light blue, and mutated residues are shown in dark blue.

FIG. 26 illustrates an example complete SSM library of DBL1_03 and related systems and methods in accordance with various embodiments disclosed herein.

As shown for FIG. 26 procedure or step 2602 (section a) is executed or otherwise implemented (e.g., by one or more processors), where as shown for FIG. 26, a structural representation of all positions targeted in the SSM library (light blue) is demonstrated. At step 2602, the four hotspot residues (red) were also targeted. Three positions were mutated in DBL1_04 (dark blue).

Further as shown for FIG. 26, procedure or step 2612 (section b) is executed or otherwise implemented (e.g., by one or more processors), where as shown for FIG. 26 an outcome of the entire SSM library is demonstrated. At step 2612, blue indicates enrichment in the binding population, while red shows enrichment in the non-binding population.

FIG. 27 illustrates an example overview of DBL2_03 SSM library and related systems and methods in accordance with various embodiments disclosed herein. As shown for FIG. 27 procedure or step 2702 (section a) is executed or otherwise implemented (e.g., by one or more processors), where as shown for FIG. 27, a structural representation of all positions targeted in the SSM library (light blue) is demonstrated. At step 2702, the four hotspot residues (red) were also targeted. Three positions were mutated in DBL2_04 (dark blue). Position 35 was not mutated in DBL_04, because all mutations in this position led to the inability of the soluble expression of the protein.

Further as shown for FIG. 27, procedure or step 2712 (section b) is executed or otherwise implemented (e.g., by one or more processors), where as shown for FIG. 27, an outcome of the entire SSM library is demonstrated. At step 2712, blue indicates enrichment in the binding population, while red shows enrichment in the non-binding population.

Further as shown for FIG. 27, procedure or step 2722 (section c) is executed or otherwise implemented (e.g., by one or more processors), where as shown for FIG. 27, binding affinities measured by SPR for the different versions of DBL2 are demonstrated.

FIG. 28 illustrates an example overview and comparison between PD-1 binders and related systems and methods in accordance with various embodiments disclosed herein. As shown for FIG. 28 procedures or steps 2802 and 2812 (sections a and b) are executed or otherwise implemented (e.g., by one or more processors), where as shown for FIG. 28, an overview and close-up of DBP40_01 (a, pink) and DBP52_01 (b, blue) models in complex with PD-1 (grey) is demonstrated. Interface seed residues similar to DBP13_01 are highlighted in red, while residues that are different are highlighted in orange.

Further as shown for FIG. 28, procedure or step 2822 (section c) is executed or otherwise implemented (e.g., by one or more processors), where as shown for FIG. 28, seeds used to design DBP13_01 (green), DBP40_01 (pink) and DBP52_01 (blue) aligned with interface residues numbered are demonstrated.

Further as shown for FIG. 28, procedure or step 2832 (section d) is executed or otherwise implemented (e.g., by one or more processors), where as shown for FIG. 28, a sequence logo of the seed interface residues for the three PD-1 binders as numbered in c is demonstrated.

FIG. 29 illustrates an example competition and specificity binding assay of DBP13_01 and DBP40_01 on the surface of yeast and related systems and methods in accordance with various embodiments disclosed herein. As shown for FIG. 29 procedure or step 2902 (section a) is executed or otherwise implemented (e.g., by one or more processors), where as shown for FIG. 29, competition between Nivolumab (green) and DBP13_01 (purple), or DBP40_01 (cyan) on the surface of PD-1 (brown) with the competing area circled is demonstrated.

Further as shown for FIG. 29, procedure or step 2912 (section b) is executed or otherwise implemented (e.g., by one or more processors), where as shown for FIG. 29, Flow cytometry histograms showing fluorescence signals on the surface of yeast displaying DBP-1. At step 2912, yeast were labeled with 500 nM PD-1-Fc (blue), 500 nM PD-1-Fc pre-incubated with 9-fold molar excess of Nivolumab Fab (orange) or labeled with secondary antibodies only (grey, Neg Ctrl).

Further as shown for FIG. 29, procedure or step 2922 (section c) is executed or otherwise implemented (e.g., by one or more processors), where as shown for FIG. 29, flow cytometry histograms showing fluorescence signal on the surface of yeast displaying DBP-1 and labeled with 500 nM pig PD1-Fc (red), 500 nM human (Hm) PD1-Fc (blue) or labeled with secondary antibodies only (grey, Neg Ctrl) are demonstrated.

FIG. 30 illustrates an example SSM of DBP13_01 and related systems and methods in accordance with various embodiments disclosed herein. As shown for FIG. 30, A full heatmap covering all positions of DBP13_01 is demonstrated. Yeast displaying point mutants were analyzed by flow cytometry and sorted for the binding and non-binding population. For each mutation and position, the log-ratio between the enrichment in binding versus non-binding was calculated. Mutations in red highlight a deleterious effect on the binding, while mutations in blue indicate a preference for the binding population.

FIG. 31 illustrates an example SSM of DBP13_01 and related systems and methods in accordance with various embodiments disclosed herein. As shown for FIG. 31 procedure or step 3102 (section a) is executed or otherwise implemented (e.g., by one or more processors), where as shown for FIG. 31, a comparison of the DBP13_01 computational model (green) and the AlphaFold (AF2) prediction (red) on the surface of PD-1 (blue) is demonstrated.

Further as shown for FIG. 31, procedure or step 3112 (section b) is executed or otherwise implemented (e.g., by one or more processors), where as shown for FIG. 31 buried interfaces in both DBP13_01 model and AF2 prediction are highlighted in red are demonstrated. At step 3112, the model interface contour is reported in yellow on the surface of the prediction as a comparison.

FIG. 32 illustrates an example surface comparison between seeds, designs and final/predicted structures and related systems and methods in accordance with various embodiments disclosed herein. As shown for FIG. 32, a comparison between each seed, design and final or predicted structure is implemented.

FIG. 33 illustrates an example surface similarity between the residues corresponding to the initial design and the final binding seed and related systems and methods in accordance with various embodiments disclosed herein. As shown for FIG. 33, each complex was aligned based on the target protein, and the surface similarity of each design to the binding seed is shown in a gradient from white to red.

Description of Various Methods in Accordance with the Disclosure Herein.

The below describes various methods in accordance with the present disclosure and various embodiments herein.

Analysis of Buried Molecular Surface Areas on Transient Interactions

A dataset of protein-protein interactions was downloaded from the PDBBind database containing all interactions with a reported affinity stronger than 10 μM; since these PPIs have a reported affinity, all were assumed to be transient. The PDBBind database does not report the chains involved in the interaction with the reported affinity; thus, for simplicity, only those complexes containing exactly two chains in the PDB crystal structure were considered for analysis.

Computing PPI Buried Surface Areas on Protein Molecular Surfaces

The MSMS program was used to compute all molecular surfaces in this work (density=3.0, water radius=1.5 Å). Since MSMS produces molecular surfaces with highly irregular meshes, meshes were regularized to a resolution of 1.0 Å using PyMESH. To compute buried surface areas of protein subunits in co-crystallized complexes, the buried surface area of the subunit was first computed, followed by the buried surface area of the complex. Any vertex in the surface of the subunit that was farther than 2.0 Å from a vertex in the complex was labeled as a buried surface vertex. The size of buried areas was computed by computing the area of each vertex labeled as a buried surface vertex.

We note that in this work we focus on the protein molecular surface (also known as solvent excluded surface) as opposed to the solvent accessible surface, which is slightly larger. In most analysis of protein-protein interactions performed in the field, the solvent accessible surface area is used to measure the buried area of PPIs, and typically this value counts the interfaces of both partners that become buried. Thus, in general the areas presented in this work are less than half of the areas reported in other work and must therefore not be used comparatively.

Decomposing Protein Surfaces into Radial Patches.

In order to process protein surface information, all molecular surfaces are decomposed into overlapping radial patches. This means that each vertex on the surface becomes the center of a radial patch of a given radius. To compute the geodesic radius of patches, throughout this work we used the Dijkstra algorithm, a fast and simple approximation to the true geodesic distance in the patch. We used a radius size of 12 Å for patches, limited to at most 200 points, which we found corresponds roughly to 400 Å² (FIG. 6a), a value close to the median size of the buried interface of transient interactions (FIG. 6b). Exceptionally, for the MaSIF-site application we limited the patch to 9 Å or 100 points to reduce the required GPU RAM for this application.

Computing the Largest Circumscribed Patch in Buried Surface Areas.

From each labeled interface point, we used the Dijkstra algorithm to compute the shortest distance to a non-interface point. The interface point with the largest distance to a non-interface point was labeled as the center of the interface, and the distance to the nearest non-interface point as the radius of the largest circumscribed patch.

Geometric and Chemical Features Used to Describe Molecular Surfaces.

Each point in a patch of the computed molecular surface was assigned an array of two geometric features (shape index, distance-dependent curvature), and three chemical features (hydrophobicity, Poisson-Boltzmann electrostatics, and a hydrogen bond potential). These features are identical to those described in Gainza et al.

Prediction of Buried Surface Area Sites in Proteins.

The MaSIF-site tool was trained to predict buried surface areas on the surface of proteins. Here, MaSIF-site was used to predict buried surface areas in each of the 27 targets of our benchmark (FIG. 7) and each of our three targets (SARS-CoV-2 RBD, PD-L1 and PD-1). MaSIF-site receives as input a protein decomposed into patches with the five previously mentioned and outputs a per-vertex regression score on the propensity of each point to become a buried surface area. In order to select binding sites in proteins, the output of MaSIF-site was decomposed into 12 Å overlapping patches, and the per-vertex prediction for all points in the patch was averaged to obtain the patch with the highest interface score. Then, iteratively, the labels of the top patch were set to zero and the process was repeated to obtain the second interface site, and so on successively.

Identifying Complementary Surfaces Using Fingerprints.

MaSIF-search was used to compute fingerprints for every overlapping patch in proteins of interest. MaSIF-search was trained on a large dataset of protein-protein interactions to receive as input the features of the target, a binder, and a random patch from potentially a different protein. MaSIF-search was trained on a Siamese architecture to produce similar fingerprints for the target patch vs. the binder patch, and dissimilar fingerprints for the target patch vs. the random patch. In order to decrease training time and improve performance, the features of the target were multiplied by −1, in order to turn the problem from one of complementarity to one of similarity.

Decomposition of the PDB into Alpha Helical Peptides.

A snapshot of the non-redundant set of the PDB was downloaded and decomposed into alpha helices, removing all non-helical elements. The DSSP program was used to label each residue according to their secondary structure. Fragments with 10 or more consecutive residues with a helical (‘H’) label assigned by DSSP were extracted. Each extracted helical fragment was treated as a monomeric protein, and surface features were computed for each one. MaSIF-search fingerprints and MaSIF-site labels were then computed for all extracted helices.

Dataset of Helix: Receptor Proteins.

The set of transient interactions from PDBBind was scanned to identify proteins that bind to helices. A protein was determined to be a helix if 80% of residues are helical and the total number of residues does not exceed 60. If both partners met the criteria, the PPI was discarded. The set was later cured to remove pairs of PPIs with high homology and finally a set of 27 unique PPIs was defined. The helices from these receptors were added to the database of alpha helical peptides; MaSIF-search fingerprints and MaSIF-site fingerprints were computed for them.

MaSIF-Seed: A Pipeline to Identify Helical Fragments in Proteins.

Based on the MaSIF-ppi-search ultra-fast searching pipeline, we developed a novel pipeline to identify potential binding seeds to targets. For each target, first MaSIF-seed was used to label each point in the surface for the propensity to form a buried surface region. Then, a fingerprint was computed for the target site. Finally, after scanning the entire protein, the best patch was selected. In one case, the SARS-CoV-2 RBD, the fourth best site was selected as it was the site with the highest potential to disrupt binding to the natural receptor. Then, a MaSIF-search fingerprint was computed for the target patch, inverting the target features before inputting them to the MaSIF-search network. The Euclidean distance between the target fingerprint and 140 million fingerprints in the helical peptide database was then computed, and all patches with a fingerprint distance <2.0 (<1.7 for RBD) were accepted.

Once fingerprints are matched, a second-stage alignment and scoring method used the RANSAC algorithm implemented in Open 3D to align the patches, similar to that presented in Gainza et al. The RANSAC algorithm implemented in Open 3D chooses three random points in the binder patch and computes the Euclidean distance of the MaSIF-search fingerprints between these points and all those points in the target patch; the most similar fingerprints provide the RANSAC algorithm with 3 correspondences to compute a transformation between the patches. Once an alignment is made a neural network was trained to classify true binders vs. non-binders. Those binders with a neural network score of more than 0.90-0.97 in the neural network score were accepted.

Clustering of Solutions.

In each design case all of the top matched seeds were clustered by first computing the root mean square deviation between all pairwise helices, computed on the C-alpha atoms of each pair of helices, in the segment overlapping over the buried surface area. The pairwise distances were then clustered using metric multi-dimensional scaling implemented in scikit-learn.

Seed Refinement.

For the PD-1 target, seed candidates proposed by MaSIF were refined using RosettaScript and a FastDesign protocol with a penalty for buried unsatisfied polar atoms in the scoring function. 33 refined seeds were selected based on the computed binding energy, the shape complementarity, the number of hydrogen bonds and the number of buried unsatisfied polar atoms.

Grafting of Seeds onto Monomeric Scaffolds and Computational Design with Rosetta.

A representative seed was selected from each solution space, and then matched using Rosetta MotifGraft to a database of 1300 monomeric scaffolds in the case of the RBD and PD-L1 designs For PD-1 designs, the 33 selected seeds were grafted to a database of 4,347 small globular proteins (<100 amino acids), originating from the PDB, two computationally designed miniprotein databaseS27 and one AF2 proteome prediction database. After grafting by Rosetta, a computational design protocol was used to design the outside of the interface for affinity to the target. Final designs were selected for yeast display based on the computed binding energy, the shape complementarity, the number of hydrogen bonds and the number of buried unsatisfied polar atoms.

Yeast Surface Display of Single Designs.

DNA sequences of binder designs were purchased from Twist Bioscience containing homology overhangs for cloning. DNA was transformed with linearized pCTcon2 (Addgene #41843) or a modified pNTA vector with V5 tag into EBY-100 yeast using the Frozen-EZ Yeast Transformation II Kit (Zymo Research). Transformed yeast were passaged once in minimal glucose medium (SDCAA) before induction of surface display in minimal galactose medium (SGCAA) overnight at 30° C. Transformed cells were washed in cold PBS with 0.05-0.1% BSA and incubated with the binding target for 2 hours at 4° C. Cells were washed once and incubated for an additional 30 minutes with appropriate antibodies. Cells were washed and analyzed using a Gallios flow cytometer (Beckman Coulter). For quantitative binding measurements, binding was quantified by measuring the fluorescence of a PE-conjugated anti-human Fc antibody (Invitrogen) detecting the Fc-fused protein target. Yeast cells were gated for the displaying population only (V5 positive).

Yeast Libraries.

Combinatorial sequence libraries were constructed by assembling multiple overlapping primers containing degenerate codons at selected positions for combinatorial sampling of the binding interface, core residues or hydrophobic surface residues. Primers were mixed (10 μM each) and assembled in a PCR reaction (55° C. annealing for 30 sec, 72° C. extension time for 1 min, 25 cycles). To amplify full-length assembled products, a second PCR reaction was performed, with forward and reverse primers specific for the full-length product. For SSM libraries and oligo pools, DNA was ordered from Twist Biosciences and amplified with primers to give homology to the pCTcon2/pNTA backbone. In all cases, the PCR product was desalted and used for transformation.

Yeast Surface Display of Libraries.

Combinatorial libraries, SSM libraries, and oligo pools were transformed as linear DNA fragments in a 5:1 ratio with linearized pCTcon2 or pNTA V5 vector as described previously into EBY-100 yeast. Transformation efficiency generally yielded around 10⁷ transformants per cuvette. Transformed yeast were passaged at least once in minimal glucose medium (SDCAA) before induction of surface display in minimal galactose medium (SGCAA) overnight at 30° C. Induced cells were labeled in the same manner as the single designs. Labeled cells were washed and sorted on a Sony SH800 cell sorter. For combinatorial libraries and oligo pool libraries, sorted cells were grown in SDCAA and prepared similarly for two additional rounds of sorting. After the third sort cells were plated on SDCAA agar and single colonies were sequenced. SSM libraries were sorted once, collecting both binding and nonbinding populations, and grown in liquid culture for plasmid preparation.

MiSeq Sequencing.

After sorting, yeast cells were grown in SDCAA medium, pelleted and plasmid DNA was extracted using Zymoprep Yeast Plasmid Miniprep II (Zymo Research) following the manufacturer's instructions. The coding sequence of the designed variants was amplified using vector-specific primer pairs, Illumina sequencing adapters and Nextera barcodes were attached using an additional overhang PCR, and PCR products were desalted with Qiaquick PCR purification kit (Qiagen) or AMPure XP selection beads (Beckman Coulter). Next generation sequencing was performed using Illumina MiSeq with appropriate read length, yielding between 0.45-0.58 million reads/sample. For bioinformatic analysis, sequences were translated in the correct reading frame, and enrichment values were computed for each sequence.

Protein Expression and Purification.

DNA sequences were ordered from Twist Bioscience and Gibson cloning or T7 ligation used to clone into bacterial (pET21b) or mammalian (pHLSec) expression vectors. Specific protein constructs and tags are listed in table AV12. Mammalian expressions were performed using the Expi293™ expression system from Thermo Fisher Scientific. Supernatant was collected 6 days post transfection, filtered, and purified. E. coli expressions were performed using BL21 (DE3) cells and IPTG induction (1 mM at OD 0.6-0.8) and growth overnight at 16-18° C. Pellets were lysed in lysis buffer (50 mM Tris, pH 7.5, 500 mM NaCl, 5% Glycerol, 1 mg/ml lysozyme, 1 mM PMSF, and 1 μg/ml DNase) with sonication, the lysate clarified, and purified. All proteins were purified using an ÄKTA pure system (GE healthcare) with either Ni-NTA affinity or protein A affinity columns followed by size exclusion chromatography. If TEV cleavage was necessary, fused proteins were dialyzed overnight at 4° C. (dialysis buffer 20 mM Tris pH 7.5, 150 mM NaCl, 10% glycerol) with excess TEV enzymes.

Surface Plasmon Resonance.

SPR measurements were performed on a Biacore 8K (GE Healthcare) with HBS-EP+ as running buffer (10 mM HEPES pH 7.4, 150 mM NaCl, 3 mM EDTA, 0.005% v/v Surfactant P20, GE Healthcare). Ligands were immobilized on a CM5 chip (GE Healthcare #29104988) via amine coupling. 500-1000 response units (RU) were immobilized and designed proteins were injected as an analyte in serial dilutions. The flow rate was 30 μl/min for a contact time of 120 s followed by 800 s dissociation time. After each injection, the surface was regenerated using 3 M magnesium chloride (for PD-L1) or 10 mM glycine, pH 3.0 (for RBD). Data were fit with 1:1 Langmuir binding model within the Biacore 8K analysis software (GE Healthcare #29310604).

Biolayer Interferometry.

BLI measurements were performed on a Gator BLI system. The running buffer was 150 mM NaCl, 10 mM HEPES pH 7.5. Fc-tagged designs were diluted to 5 ug/mL and immobilized on the tips (1-2 nm immobilized). The loaded tips were then dipped into serial dilutions of either spike protein or RBD. Curves were fit using a 1:1 model on the Gator software after subtracting the background.

Size Exclusion Chromatography Multi-Angle Light Scattering (SEC-MALS).

Size exclusion chromatography with an online multi-angle light scattering device (miniDAWN TREOS, Wyatt) was used to determine the oligomeric state and molecular weight for the protein in solution. Purified proteins were concentrated to 1 mg/ml in PBS (pH 7.4), and 100 μl of the sample was injected into a Superdex 75 300/10 GL column (GE Healthcare) with a flow rate of 0.5 ml/min, and UV₂₈₀ and light scattering signals were recorded. Molecular weight was determined using the ASTRA software (version 6.1, Wyatt).

Circular Dichroism.

Far-UV circular dichroism spectra were measured using a Chirascan™ spectrometer (AppliedPhotophysics) in a 1-mm path-length cuvette. The protein samples were prepared in a 10 mM sodium phosphate buffer at a protein concentration between 20 and 50 μM. Wavelengths between 200 nm and 250 nm were recorded with a scanning speed of 20 nm min⁻¹ and a response time of 0.125 secs. All spectra were averaged two times and corrected for buffer absorption. Temperature ramping melts were performed from 20 to 90 □C with an increment of 2 ≡C/min. Thermal denaturation curves were plotted by the change of ellipticity at the global curve minimum to calculate the melting temperature (Tₘ).

Purification of Proteins for Crystallography.

PD-L1 extracellular domain fragment (from F19 to R238) was over-expressed as inclusion bodies in the BL21 (DE3) strain of E. coli. Renaturation and purification of PD-L1 was performed as previously described. Briefly, inclusion bodies of PD-L1 was diluted against a refolding buffer (100 mM Tris, pH 8.0; 400 mM L-Arginine; 5 mM EDTA-Na; 5 mM Glutathione (GSH); 0.5 mM Glutathione disulfide (GSSG)) at 4° C. for 24 h. Then the PD-L1 was concentrated and exchanged into a buffer of 20 mM Tris-HCl (pH 8.0) and 15 mM NaCl and further analyzed by HiLoad 16/60 Superdex 75 pg (Cytiva) chromatography.

PD-L1 binder designs, DBL1_03 and DBL2-02, were over expressed in E. coli as inclusion bodies. Renaturation and purification of the PD-L1 binder designs was performed as the PD-L1 protein. PD-L1 and binder designs were then mixed together at a molar ratio of 1:2 and incubated for 1 h on ice. The binder/PD-L1 complex was further purified by HiLoad 16/60 Superdex 75 pg (Cytiva) chromatography.

Data Collection and Structure Determination.

For crystal screening, 1 μl of binder/PD-L1 complex protein solution (10 mg/mL) was mixed with 1 μl of crystal growing reservoir solution. The resulting mixture was sealed and equilibrated against 100 μl of reservoir solution at 4° or 18° C. Crystals of DBL1_03/PD-L1 complex were grown in 0.2 M potassium/sodium tartrate, 0.1 M Bis Tris propane, pH 6.5, 20 w/v PEG 3350, and crystals of DBL2_02/PD-L1 complex were grown in. 0.1 M Sodium citrate tribasic dihydrate pH 5.5, 16% w/v Polyethylene glycol 8,000. Crystals were flash-cooled in liquid nitrogen after incubating in anti-freezing buffer (reservoir solution containing 20% (v/v) glycerol). Diffraction data of crystals were collected at Shanghai Synchrotron Radiation Facility (SSRF) BL19U. The collected intensities were subsequently processed and scaled using the Denzo program and the HKL2000 software package (HKL Research). The structures were determined using molecular replacement with the program Phaser MR in CCP4, with the reported PD-L1 structure (PDB: 3RRQ) as the search model. COOT and PHENIX were used for subsequent model building and refinement. The stereochemical qualities of the final model were assessed with MolProbit. Structure-related figures were generated using PyMOL.

Luminex Binding Assays.

Luminex beads were prepared as previously published. Briefly, MagPlex beads were covalently coupled to SARS-CoV-2 spike proteins of different variants. The serial dilutions of the antibodies or design were performed and binding curves were calculated as previously published. Response curves were fit using GraphPad Prism nonlinear four parameter curve fitting analysis of the log(agonist) versus response.

Live Virus Neutralization Assays.

The virus neutralization assays were performed as previously published. Briefly, VeroE6 cells were seeded in 96 well plates the day before the infection. The DBR3_03-Fc compound in serial dilutions was mixed with omicron virus and incubated at 37° C. for one hour before addition to the cells. The cells with virus were kept a further 48 hours at 37° C., then washed and fixed for crystal violet staining and analysis. Neutralization EC50 calculations were performed using GraphPad Prism nonlinear four parameter curve fitting analysis.

Cryo-EM Sample Preparation and Data Acquisition.

The carbon/copper grids (Quantifoil R2/1, 400 mesh) were glow-discharged at 15 mA for 30 seconds. Respectively, 3.0 μl aliquot at concentration of 0.87 or 1.0 mg/ml of the spike_{D614G}-binder sample and spike_{Omicron}-binder sample were applied onto the grids and blotted for 4.0-8.0 s, then flash-frozen in a pre-cooling liquid ethane/propane mixture using Vitrobot Mark IV (Thermo Fisher Scientific) with 100% humidity, which operated at 4° C.

The spike_{D614G}-binder data composed of 20,794 movies was collected on a Titan Krios G4 microscope (Cold FEG, Thermo Fisher Scientific), operated at 300 kV and equipped with Falcon4 direct detection camera (Thermo Fisher Scientific). Movies were recorded in counting mode using automation program of EPU (Thermo Fisher Scientific) with a physical pixel size of 0.40 Å per pixel and the defocus ranging from −0.8 to −2.0 μm. Exposures were calibrated to 60 e-/Å2 total dose. For spike_{Omicron}-binder data, 22,266 movies were recorded on a Titan Krios G4 microscope, equipped with SelectrisX imaging filter (Thermo Fisher Scientific) containing a Falcon4 direct detection camera. Exposures were adjusted to 60 e-/Å2 total dose with the physical pixel size of 0.726 Å per pixel and a defocus range of −0.8 to −2.5 μm. The raw movies were exported as EER format.

Cryo-EM Image Processing, Model Building and Refinement.

Details of the image processing are shown in FIGS. 14-19. All the movies were imported into cryoSPARC v3.3.1 and gain-normalized, motion-corrected and dose-weighted using the cryoSPARC implementation of patch-based motion correction.

CTF Estimation was performed using the patch-based option in cryoSPARC. For the sample of spike_{D614G} in complex with the de-novo designed binder, 832,816 particles were automatically picked by template picker and followed by three rounds of 2D classifications, resulting in a particle set of 184,763 particles. With the ab-Initio and hetero-Refine implementation in cryoSPARC, the particles were grouped into three classes. The best 3D class composed of 97,804 particles were further subjected to another round of ab-Initio reconstruction and hetero-Refinement. The well-resolved class consisting of 50,448 particles resulted in a 2.63 Å overall resolution global map in Cl symmetry. The binder-RBD region was refined with soft mask, resulting in a local map at 3.10 Å resolution. Resolution of all the 3D maps was estimated based on the Fourier shell correlation (FSC) with a cutoff value of 0.143. For the data processing of the spike_{Omicron}-binder complex sample, 1,820,333 particles were picked by using cryoSPARC template-based implementation. After two rounds of 2D classifications, 981,561 particles were selected and then subjected to ab-Initio reconstruction and hetero-Refinement, resulting in a set of 595,599 particles. Subsequently, the selected particle set was classified by multiple rounds of 3D classifications in cryoSPARC. The best-resolved 3D class containing 50,758 particles resulted in a 2.80 Å overall resolution map and the binder-RBD region was further improved by performing the focused refinement with a soft mask and had a 3.29 Å resolution as estimated by FSC at 0.143 cutoff.

For model building of spike_{D614G}-binder, the previous model (PDB: 7BNO, spike_{D614G}) was used for the region of spike_{D614G} as a starting model. Model was rigid-body fit into resulted cryo-EM density in UCSF Chimera and adjusted manually in Coot 0.9.4. De novo building for the binder parts were performed manually in Coot 0.9.4. For the building of spike_{Omicron}-binder structure, the model (PDB: 7QO7, spike_{Omicron}) was fit into the density and rebuilt, extended manually using UCSF Chimera and Coot 0.9.4. After the structural rebuilding, all the atomic models were refined using the Phenix (1.19.2-4158) implementation of Real. Space. refine with general restraintS7. EM densities and atomic models were visualized in UCSF Chimera, UCSF ChimeraX and Pymol.

Data Availability.

Cryo-EM maps for spike_{D614G}-binder full, spike_{D614G}-binder local, spike_{Omicron}-binder full and spike_{Omicron}-binder local were deposited in the Electron Microscopy Data Bank respectively under the codes of EMD-14947 (spike(D614G)-binder full and spike(D614G)-binder local maps), EMD-14922 (spike(Omicron)-binder full) and EMD-14930 (spike(Omicron)-binder local). Atomic models were deposited at the PDB under the following accession codes: 7ZSS (spike(D614G)-binder), 7ZRV (spike(Omicron)-binder full) and 7ZSD (spike(Omicron)-binder local). Crystal structures have been deposited at the PDB under the following accession codes: 7XYQ (DBL1_03-PD-L1 complex) and 7XAD (DBL2_02-PD-L1 complex).

Additional Aspects of the Disclosure.

Although some aspects have been described in the context of a system, apparatus, and/or method it is clear that these aspects also represent a description of the corresponding method, where a block or device corresponds to a method step or a feature of a method step. Analogously, aspects described in the context of a method step also represent a description of a corresponding block or item or feature of a corresponding apparatus.

Embodiments of the invention may be implemented on a computer system. The computer system may be a local computer device (e.g. personal computer, laptop, tablet computer or mobile phone) with one or more processors and one or more storage devices or may be a distributed computer system (e.g. a cloud computing system with one or more processors and one or more storage devices distributed at various locations, for example, at a local client and/or one or more remote server farms and/or data centers). The computer system may comprise any circuit or combination of circuits. In one embodiment, the computer system may include one or more processors which can be of any type. As used herein, processor may mean any type of computational circuit, such as but not limited to a microprocessor, a microcontroller, a complex instruction set computing (CISC) microprocessor, a reduced instruction set computing (RISC) microprocessor, a very long instruction word (VLIW) microprocessor, a graphics processor, a digital signal processor (DSP), multiple core processor, a field programmable gate array (FPGA), or any other type of processor or processing circuit. Other types of circuits that may be included in the computer system may be a custom circuit, an application-specific integrated circuit (ASIC), or the like, such as, for example, one or more circuits (such as a communication circuit) for use in wireless devices like mobile telephones, tablet computers, laptop computers, two-way radios, and similar electronic systems. The computer system may include one or more storage devices, which may include one or more memory elements suitable to the particular application, such as a main memory in the form of random access memory (RAM), one or more hard drives, and/or one or more drives that handle removable media such as compact disks (CD), flash memory cards, digital video disk (DVD), and the like. The computer system may also include a display device, one or more speakers, and a keyboard and/or controller, which can include a mouse, trackball, touch screen, voice-recognition device, or any other device that permits a system user to input information into and receive information from the computer system.

Some or all of the method steps may be executed by (or using) a hardware apparatus, like for example, a processor, a microprocessor, a programmable computer or an electronic circuit. In some embodiments, some one or more of the most important method steps may be executed by such an apparatus.

Depending on certain implementation requirements, embodiments of the invention can be implemented in hardware or in software. The implementation can be performed using a non-transitory storage medium such as a digital storage medium, for example a floppy disc, a DVD, a Blu-Ray, a CD, a ROM, a PROM, and EPROM, an EEPROM or a FLASH memory, having electronically readable control signals stored thereon, which cooperate (or are capable of cooperating) with a programmable computer system such that the respective method is performed. Therefore, the digital storage medium may be computer readable.

Some embodiments according to the invention comprise a data carrier having electronically readable control signals, which are capable of cooperating with a programmable computer system, such that one of the methods described herein is performed.

Generally, embodiments of the present invention can be implemented as a computer program product with a program code, the program code being operative for performing one of the methods when the computer program product runs on a computer. The program code may, for example, be stored on a machine readable carrier.

Other embodiments comprise the computer program for performing one of the methods described herein, stored on a machine readable carrier.

In other words, an embodiment of the present invention is, therefore, a computer program having a program code for performing one of the methods described herein, when the computer program runs on a computer.

A further embodiment of the present invention is, therefore, a storage medium (or a data carrier, or a computer-readable medium) comprising, stored thereon, the computer program for performing one of the methods described herein when it is performed by a processor. The data carrier, the digital storage medium or the recorded medium are typically tangible and/or non-transitionary. A further embodiment of the present invention is an apparatus as described herein comprising a processor and the storage medium.

A further embodiment of the invention is, therefore, a data stream or a sequence of signals representing the computer program for performing one of the methods described herein. The data stream or the sequence of signals may, for example, be configured to be transferred via a data communication connection, for example, via the internet.

A further embodiment comprises a processing means, for example, a computer or a programmable logic device, configured to, or adapted to, perform one of the methods described herein.

A further embodiment comprises a computer having installed thereon the computer program for performing one of the methods described herein.

A further embodiment according to the invention comprises an apparatus or a system configured to transfer (for example, electronically or optically) a computer program for performing one of the methods described herein to a receiver. The receiver may, for example, be a computer, a mobile device, a memory device or the like. The apparatus or system may, for example, comprise a file server for transferring the computer program to the receiver.

In some embodiments, a programmable logic device (for example, a field programmable gate array) may be used to perform some or all of the functionalities of the methods described herein. In some embodiments, a field programmable gate array may cooperate with a microprocessor in order to perform one of the methods described herein. Generally, the methods are preferably performed by any hardware apparatus.

Embodiments may be based on using a machine-learning model or machine-learning algorithm. Machine learning may refer to algorithms and statistical models that computer systems may use to perform a specific task without using explicit instructions, instead relying on models and inference. For example, in machine-learning, instead of a rule-based transformation of data, a transformation of data may be used, that is inferred from an analysis of historical and/or training data. For example, the content of images may be analyzed using a machine-learning model or using a machine-learning algorithm. In order for the machine-learning model to analyze the content of an image, the machine-learning model may be trained using training images as input and training content information as output. By training the machine-learning model with a large number of training images and/or training sequences (e.g. words or sentences) and associated training content information (e.g. labels or annotations), the machine-learning model “learns” to recognize the content of the images, so the content of images that are not included in the training data can be recognized using the machine-learning model. The same principle may be used for other kinds of sensor data as well: By training a machine-learning model using training sensor data and a desired output, the machine-learning model “learns” a transformation between the sensor data and the output, which can be used to provide an output based on non-training sensor data provided to the machine-learning model. The provided data (e.g. sensor data, metadata and/or image data) may be preprocessed to obtain a feature vector, which is used as input to the machine-learning model.

Machine-learning models may be trained using training input data. The examples specified above use a training method called “supervised learning”. In supervised learning, the machine-learning model is trained using a plurality of training samples, wherein each sample may comprise a plurality of input data values, and a plurality of desired output values, i.e. each training sample is associated with a desired output value. By specifying both training samples and desired output values, the machine-learning model “learns” which output value to provide based on an input sample that is similar to the samples provided during the training. Apart from supervised learning, semi-supervised learning may be used. In semi-supervised learning, some of the training samples lack a corresponding desired output value. Supervised learning may be based on a supervised learning algorithm (e.g. a classification algorithm, a regression algorithm or a similarity learning algorithm. Classification algorithms may be used when the outputs are restricted to a limited set of values (categorical variables), i.e. the input is classified to one of the limited set of values. Regression algorithms may be used when the outputs may have any numerical value (within a range). Similarity learning algorithms may be similar to both classification and regression algorithms but are based on learning from examples using a similarity function that measures how similar or related two objects are. Apart from supervised or semi-supervised learning, unsupervised learning may be used to train the machine-learning model. In unsupervised learning, (only) input data might be supplied and an unsupervised learning algorithm may be used to find structure in the input data (e.g. by grouping or clustering the input data, finding commonalities in the data). Clustering is the assignment of input data comprising a plurality of input values into subsets (clusters) so that input values within the same cluster are similar according to one or more (pre-defined) similarity criteria, while being dissimilar to input values that are included in other clusters.

Reinforcement learning is a third group of machine-learning algorithms. In other words, reinforcement learning may be used to train the machine-learning model. In reinforcement learning, one or more software actors (called “software agents”) are trained to take actions in an environment. Based on the taken actions, a reward is calculated. Reinforcement learning is based on training the one or more software agents to choose the actions such, that the cumulative reward is increased, leading to software agents that become better at the task they are given (as evidenced by increasing rewards).

Furthermore, some techniques may be applied to some of the machine-learning algorithms. For example, feature learning may be used. In other words, the machine-learning model may at least partially be trained using feature learning, and/or the machine-learning algorithm may comprise a feature learning component. Feature learning algorithms, which may be called representation learning algorithms, may preserve the information in their input but also transform it in a way that makes it useful, often as a pre-processing step before performing classification or predictions. Feature learning may be based on principal components analysis or cluster analysis, for example.

In some examples, anomaly detection (i.e. outlier detection) may be used, which is aimed at providing an identification of input values that raise suspicions by differing significantly from the majority of input or training data. In other words, the machine-learning model may at least partially be trained using anomaly detection, and/or the machine-learning algorithm may comprise an anomaly detection component.

In some examples, the machine-learning algorithm may use a decision tree as a predictive model. In other words, the machine-learning model may be based on a decision tree. In a decision tree, observations about an item (e.g. a set of input values) may be represented by the branches of the decision tree, and an output value corresponding to the item may be represented by the leaves of the decision tree. Decision trees may support both discrete values and continuous values as output values. If discrete values are used, the decision tree may be denoted a classification tree, if continuous values are used, the decision tree may be denoted a regression tree.

Association rules are a further technique that may be used in machine-learning algorithms. In other words, the machine-learning model may be based on one or more association rules. Association rules are created by identifying relationships between variables in large amounts of data. The machine-learning algorithm may identify and/or utilize one or more relational rules that represent the knowledge that is derived from the data. The rules may e.g. be used to store, manipulate or apply the knowledge.

Machine-learning algorithms are usually based on a machine-learning model. In other words, the term “machine-learning algorithm” may denote a set of instructions that may be used to create, train or use a machine-learning model. The term “machine-learning model” may denote a data structure and/or set of rules that represents the learned knowledge (e.g. based on the training performed by the machine-learning algorithm). In embodiments, the usage of a machine-learning algorithm may imply the usage of an underlying machine-learning model (or of a plurality of underlying machine-learning models). The usage of a machine-learning model may imply that the machine-learning model and/or the data structure/set of rules that is the machine-learning model is trained by a machine-learning algorithm.

For example, the machine-learning model may be an artificial neural network (ANN). ANNs are systems that are inspired by biological neural networks, such as can be found in a retina or a brain. ANNs comprise a plurality of interconnected nodes and a plurality of connections, so-called edges, between the nodes. There are usually three types of nodes, input nodes that receiving input values, hidden nodes that are (only) connected to other nodes, and output nodes that provide output values. Each node may represent an artificial neuron. Each edge may transmit information, from one node to another. The output of a node may be defined as a (non-linear) function of its inputs (e.g. of the sum of its inputs). The inputs of a node may be used in the function based on a “weight” of the edge or of the node that provides the input. The weight of nodes and/or of edges may be adjusted in the learning process. In other words, the training of an artificial neural network may comprise adjusting the weights of the nodes and/or edges of the artificial neural network, i.e. to achieve a desired output for a given input.

Alternatively, the machine-learning model may be a support vector machine, a random forest model or a gradient boosting model. Support vector machines (i.e. support vector networks) are supervised learning models with associated learning algorithms that may be used to analyze data (e.g. in classification or regression analysis). Support vector machines may be trained by providing an input with a plurality of training input values that belong to one of two categories. The support vector machine may be trained to assign a new input value to one of the two categories. Alternatively, the machine-learning model may be a Bayesian network, which is a probabilistic directed acyclic graphical model. A Bayesian network may represent a set of random variables and their conditional dependencies using a directed acyclic graph. Alternatively, the machine-learning model may be based on a genetic algorithm, which is a search algorithm and heuristic technique that mimics the process of natural selection.

What is claimed is:

1. A computer-implemented method for protein interaction design using surface fingerprints, the method comprising:
predicting at least one target interface site with high binding propensity;
identifying at least one binding seed that displays required features to engage the target site; and
performing a binding seed transplantation to protein scaffolds to confer stability and additional contacts on the designed interface.

2. The computer-implemented method of claim 1, wherein the at least one target interface site comprises at least one target buried interface site, and wherein predicting the target buried interface site comprises generating at least one surface fingerprint associated with a protein interaction based on at least one protein interface.

3. The computer-implemented method of claim 2, wherein the at least one surface fingerprint embeds geometric and/or chemical features of molecular surfaces.

4. The computer-implemented method of claim 1, further comprising performing a surface fingerprint-based search.

5. The computer-implemented method of claim 1, further comprising decomposing a protein molecular surface into overlapping radial patches, preferably with a radius of 12 Å.

6. The computer-implemented method of claim 1, further comprising computing a coordinate system in geodesic space.

7. The computer-implemented method of claim 1, further comprising using a first machine-learning model to output vector fingerprint descriptors that are complementary between interacting protein pairs and dissimilar between non-interacting pairs.

8. The computer-implemented method of claim 1, further comprising using a second machine-learning model to score matching surface patches, in particular for computing an interface post-alignment score.

9. The computer-implemented method of claim 1, wherein the at least one target interface site with high binding propensity is a prediction output by a machine-learning model trained on data comprising surface fingerprints and/or geometric or chemical features of molecular surfaces.

10. The computer-implemented method of claim 1, wherein the binding seed and/or the binding seed transplantation is used for one or more of:
obtaining a protein-based therapeutic;
obtaining an antibody;
obtaining an inhibitor; or
obtaining a vaccine.

11. A system comprising:
one or more processors,
a memory; and
computing instructions stored in the memory and configured to execute on the one or more processors, which when executed, causes the one or more processors to:
predict at least one target interface site with high binding propensity,
identifying at least one binding seed that displays required features to engage the target site, and
performing a binding seed transplantation to protein scaffolds to confer stability and additional contacts on the designed interface.

12. The system of claim 11, wherein the computing instructions are further configured, when executed by the one or more processors, to predict at least one target buried interface site comprises generating at least one surface fingerprint associated with a protein interaction based on at least one protein interface,

13. The system of claim 12, wherein the at least one surface fingerprint embeds geometric and/or chemical features of molecular surfaces.

14. The system of claim 11, wherein the computing instructions are further configured, when executed by the one or more processors, to perform a surface fingerprint-based search.

15. The system of claim 11, wherein the computing instructions are further configured, when executed by the one or more processors, to decompose a protein molecular surface into overlapping radial patches, preferably with a radius of 12 Å.

16. The system of claim 11, wherein the computing instructions are further configured, when executed by the one or more processors, to compute a coordinate system in geodesic space.

17. The system of claim 11, wherein the computing instructions are further configured, when executed by the one or more processors, to execute a first machine-learning model to output vector fingerprint descriptors that are complementary between interacting protein pairs and dissimilar between non-interacting pairs.

18. The system of claim 11, wherein the computing instructions are further configured, when executed by the one or more processors, to execute a second machine-learning model to score matching surface patches, in particular for computing an interface post-alignment score.

19. The system of claim 11, wherein the at least one target interface site with high binding propensity is a prediction output by a machine-learning model trained on data comprising surface fingerprints and/or geometric or chemical features of molecular surfaces.

20. A computer-readable medium storing a computer program, the computer program comprising instructions which, when the program is executed by one or more processors, cause the one or more processors to:
predict at least one target interface site with high binding propensity;
identifying at least one binding seed that displays required features to engage the target site; and
performing a binding seed transplantation to protein scaffolds to confer stability and additional contacts on the designed interface.
